(19) United States
(12) Patent Application Publication
(10) Pub. No.: US 2007/0117173 A1
(43) Pub. Date: May 24, 2007

(54) Stable storage of proteins

(76) Inventors: Peter Rognvald Levison, Havant (GB); David John Harry Smith, Fleet (GB)

Correspondence Address: EDWARDS & ANGELL, LLP, P.O. BOX 55874, BOSTON, MA, 02205, US

(21) Appl. No.: 10/488,849

(22) PCT Filed: Sep. 5, 2002
(86) PCT No.: PCT/GB02/04048

§ 371 Date: Oct. 7, 2004

(30) Foreign Application Priority Data
Sep. 5, 2001 (GB) 01214816
Oct. 29, 2001 (GB) 01259472
Mar. 5, 2002 (GB) 02051266

(51) Int. Cl. C12Q 1/37; C12M 1/34

(52) U.S. Cl. 435/23; 435/287.1

(57) ABSTRACT

The present invention provides a method of stably storing a protein, the method comprising applying a protein to be stored to a substrate which has been treated with a polyhydric compound and dried, wherein the amount of the polyhydric compound present in the substrate is sufficient to stabilise the protein, and wherein the substrate does not consist of glass. In one embodiment the protein to be stored is trypsin.

### FIELD OF THE INVENTION

The present invention relates generally to the stable storage of proteins on a substrate treated with a polyhydric compound and dried. In one embodiment, the invention relates to the storage of blood proteins, whole blood and blood fractions, in particular plasma proteins, serum proteins and complement.

### BACKGROUND TO THE INVENTION

Proteins are the products of active genes and are responsible for biochemical function in organisms. The study of protein function is gaining importance with the emergence of proteomics. Central to protein function is the maintenance of the 3-dimensional structure of protein molecules throughout their isolation from their host system.

There is often a need to store a protein-containing sample for a finite period of time. During this period protein denaturation may occur. Many proteins are labile and consequently storage methods may vary. One generic approach for protein storage, which is widely used in the biochemicals supply and biopharmaceuticals industries, is lyophilisation (also known as freeze-drying). This method of removing water molecules can stabilise some proteins but it is costly, time-consuming and instrument-dependent and can lead to irreversible denaturation of some proteins.

There is therefore a need for a method of protein storage which is available to use in most laboratories without specialised equipment, and which can handle protein samples at various levels of purity and which can be used to store proteins for a period of time in a medium from which the protein can be removed for subsequent application such as purification and/or analysis.

U.S. Pat. No. 5,155,024 describes an analytical element, having a peroxidase-labeled ligand analog uniformly distributed within a water-soluble binder composition comprising at least 50% by weight of an unspecified poly(vinyl alcohol) (PVA). As a result, the peroxidase is said to retain more of its stability prior to use.

EP 0,304,163 also describes an analytical element, having a peroxidase-labeled ligand analog uniformly distributed within a layer comprising an unspecified PVA. The layer further comprises glycerol which, in combination with the PVA, is said to further aid the stabilisation of the peroxidase-labeled ligand analog.

Both U.S. Pat. No. 5,155,024 and EP 0,304,163 teach that the peroxidase-labeled ligand analog must be uniformly distributed within the PVA-containing layer. To achieve such a uniform distribution, the peroxidase-labeled ligand analog must be mixed with the PVA composition prior to the application of the peroxidase-labeled ligand analog to the carrier matrix. The analytical element is thus supplied to customers with the peroxidase-labeled ligand analog pre-bound onto the carrier matrix so that it may be used to assay liquids such as biological fluids.

U.S. Pat. No. 5,403,706 discloses glass carrier matrices dissolvably impregnated with a reagent such as an aqueous protein solution. One method for preparing a PVA-coated glass fibre-fleece is to treat a previously prepared glass fibre fleece with a solution of PVA in water or appropriate organic solvent and to then dry the matrix. It is stated that such treatment should be carried out at a temperature above 60° C. Another method is to mix PVA powder or fibres to a pulp of glass fibres and to dissolve or melt the PVA so that the PVA forms a complete and uniform coating on the glass fibres.

The teaching of U.S. Pat. No. 5,403,706 is restricted to glass papers. Indeed, U.S. Pat. No. 5,403,706 teaches in order to achieve advantageous properties for the carrier matrix, said to be the stabilisation of impregnated reagents even after comparatively long storage and even after storage at an elevated temperature, the carrier matrix must comprise two components, the first being glass and the second being the PVA composition. Further, U.S. Pat. No. 5,403,706 specifically teaches against the use of paper fleeces since it is said either that they do not bind the applied reagent sufficiently well so that, ever during storage, a part of the applied reagent is detached or that the binding of the reagent is so strong that it cannot be eluted quickly and completely.

U.S. Pat. No. 5,118,609 describes a carrier fleece for dissolvably impregnated reagents. The carrier fleece is said to help stabilise the reagents allowing a comparatively long storage of the reagents. U.S. Pat. No. 5,118,609 teaches that the carrier fleece must consist of three components, namely fibres based on cellulose (5 to 60% by weight), polymers based on polyester and/or polyamide (40 to 95% by weight), and an organic binding agent which has hydroxyl and/or ester groups (5 to 30% by weight).

As mentioned above, the study of protein function is gaining importance with the emergence of proteomics. Central to proteomics is the digestion of proteins (e.g. by trypsin) either as a crude or enriched proteome sample or as an excised gel spot following electrophoresis, typically 2-dimensional electrophoresis. The tryptic digestion process from a gel typically involves excision of the gel spot using a punch, dehydration of the gel plug and subsequent rehydration in the trypsin solution. This process is considered far from ideal and can result in poor release of proteins/peptides from the gel, low recoveries due to their adsorption to the walls of the lysis chamber, typically plastic, and dilute tryptic digests. An alternative approach is electroelution where the proteins are eluted from the gel by an electric field.

### SUMMARY OF THE INVENTION

The invention is based on the discovery that proteins can be stably stored by impregnation of the protein onto a variety of non-glass substrates which have been treated with a polyhydric compound, such as PVA. The discovery is surprising since U.S. Pat. No. 5,155,024 and EP 0,304,163 both teach that the protein to be stabilised (a peroxidase-labeled ligand analog) must be uniformly distributed within a PVA composition for stabilisation to be effective. Further, it has now been shown that, contrary to U.S. Pat. No. 5,403,706, a non-glass substrate (e.g. a cellulose substrate) may be used in conjunction with PVA for the stable storage of proteins. Similarly, it has now been shown that, contrary to U.S. Pat. No. 5,118,609 that stabilisation may be achieved in the absence of polymers based on polyester and/or polyamide.

Accordingly, a first aspect of the. invention provides a method of stably storing a protein, the method comprising applying a protein to be stored to a substrate which has been treated with a polyhydric compound and dried, wherein the amount of the polyhydric compound present in the substrate is sufficient to stabilise the activity of the protein, and wherein the substrate does not consist of glass.

The protein to be stored may be present in a suitable medium, such as a solution, gel or macerated gel plug suspension which may then be applied to the substrate.

Accordingly, one embodiment of the first aspect of the invention provides a method of stably storing a protein, the method comprising applying a solution of the protein to be stored to a substrate which has been treated with a polyhydric compound and dried, wherein the amount of the polyhydric compound present in the substrate is sufficient to stabilise the activity of the protein, and wherein the substrate does not consist of glass.

The expressions “stabilise the protein” and “stabilise the activity of the protein” (which expressions are used interchangeably herein) mean that the protein exhibits an activity half life which is greater than the activity half life of the same protein, under the same conditions, applied to the substrate absent treatment with the polyhydric compound. The expression “activity half life” means the time period for which the protein retains at least 50% of the activity of the protein exhibited immediately after its application to the substrate. The activity half life of the protein depends on the physical properties of the protein. Thus, where more than one type of protein is stored, the proteins may be stabilised for different periods of time. Preferably the activity half life is extended more than 2-, 5-, 10-, 50-, 100-, 500-, 1000-, 5000- or 10000- fold. Preferably, the activity life of the protein when stored on a substrate of the invention is greater than 0.5, 1, 2, 3 or 6 hours. Preferably, the activity half life of the protein is greater than 12 hours, more preferably greater than 24 hours, more preferably greater than 48 hours, more preferably greater than one week, more preferably greater than two weeks, most preferably greater than three weeks. Preferably, one or two weeks after its application to the substrate, the protein retains at least 60%, 70%, 75%, 80%, 85%, 90% or 95% of its activity when compared to the activity of the protein immediately after its application to the substrate. Preferably, three, four, five, six, seven, eight, nine, ten, eleven, twelve, fifteen, twenty, thirty, forty, or fifty weeks after its application to the substrate, the protein retains at least 60%, 70%, 75%, 80%, 85%, 90% or 95% of its activity when compared to the activity of the protein immediately after its application to the substrate.

Preferably, the amount of the polyhydric compound is sufficient to stabilise the protein for at least one, two or three weeks. Preferably, the amount of the polyhydric compound is sufficient to stabilise the protein (e.g. under ambient conditions at room temperature, e.g. at 20±5° C. and 50-70% RH, e.g. at 22.5° C. and 60% RH). for at least four, five, six, seven, eight, nine, ten, eleven, twelve, fifteen, eighteen, twenty, twenty-five, thirty, forty, fifty, sixty or seventy weeks,

By a protein which is stabilised for at least one, two, three, four, five, six, seven, eight, nine, ten, eleven, twelve, fifteen, eighteen, twenty, twenty-five, thirty, forty, fifty, sixty or seventy week, we refer to a protein which, one, two, three, four, five, six, seven, eight, nine, ten, eleven, twelve, fifteen, eighteen, twenty, twenty-five, thirty, forty, fifty, sixty or seventy weeks respectively after its application to the substrate, retains at least 50% of its activity when compared to the activity of the protein immediately after its application to the substrate. Further, as will be appreciated from the above, the protein will exhibit an activity half life which is greater than the activity half life of the same protein, under the same conditions, applied to the substrate absent treatment with the polyhydric compound.

Preferably, one, two or three weeks after its application to the substrate, the protein retains at least 60%, 70%, 75%, 80%, 85%, 90% or 95% of its activity when compared to the activity of the protein immediately after its application to the substrate. Preferably, four, five, six, seven, eight, nine, ten, eleven, twelve, fifteen, eighteen, twenty, twenty-five, thirty, forty, fifty, sixty or seventy weeks after its application to the substrate, the protein retains at least 60%, 70%, 75%, 80%, 85%, 90% or 95% of its activity when compared to the activity of the protein immediately after its application to the substrate.

Where we refer to a protein retaining a certain percentage of its activity after a period of storage on a substrate of the invention, it is preferred that the protein is stored on the substrate under ambient conditions at room temperature.

Preferably, the protein to be stored is stored on the paper (or other substrate) for at least 0.5, one, two, three, four, five, six, seven, eight, nine, ten or eleven hours. Preferably, the protein to be stored is stored on the paper (or other substrate) for at least 0.5, one, two, three, four, five or six days. Preferably, the protein to be stored is stored on the paper (or other substrate) for at least one, two or three weeks. Preferably, the protein to be stored is stored on the paper (or other substrate) for at least three, four, five, six, seven, eight, nine, ten, eleven, twelve, fifteen, eighteen, twenty, twenty-five, thirty, forty, fifty, sixty or seventy weeks.

Those skilled in the art will be able to establish the amount of the polyhydric compound which must be present to provide for stabilisation of the protein for at least three four, five, six, seven, eight, nine, ten, eleven, twelve, fifteen, eighteen, twenty, twenty-five, thirty, forty, fifty, sixty or seventy weeks, in addition to the other time periods mentioned above. Those skilled in the art will further appreciate that the ability to store a protein for a prolonged time period such as eighteen weeks will at least in part depend on the physical properties of the protein.

The terms “polypeptide” and “protein” are used interchangeably and refer to any polymer of amino acids (dipeptide or greater) linked through peptide bonds or modified peptide bonds. Thus, the terms “polypeptide” and “protein” include oligopeptides, protein fragments, fusion proteins and the like. It should be appreciated that the terms “polypeptide” and “protein”, as used herein, includes moieties such as lipoproteins and glycoproteins.

Frequently, it is desirable to digest proteins with one or more proteinases or to subject proteins to some other treatment which may impair or abolish protein activity. Thus, the proteins resulting from such treatment may or may not retain activity. While the proteins may not retain activity, it may nevertheless be useful to store such proteins on a polyhydric-treated-substrate of the present invention. For instance, it may be desirable to preserve the structural integrity of any protein fragments obtained by treating a protein with a proteinase enzyme. Similarly, it may be desirable to synthesise proteins which do not have activity as such and to store the protein in an environment where chemical degradation of the protein is inhibited.

Accordingly, a second aspect of the invention provides a method of stably storing a protein, the method comprising applying a protein to be stored to a substrate which has been treated with a polyhydric compound and dried, wherein the amount of the polyhydric compound present in the substrate is sufficient to chemically stabilise the protein, and wherein the substrate does not consist of glass.

The term “chemically stabilise the protein” (as distinct from “stabilise the protein” and “stabilise the activity of the protein”) is used herein to refer to the stabilisation of proteins against chemical degradation, such as by hydrolysis. The protein stored in accordance with the second aspect of the invention may or may not have inherent biological activity. Preferably, the protein does not possess inherent biological activity.

Preferably, the protein to be stored has been subjected to proteinase treatment or to some other treatment which may impair or abolish the activity of the protein(s).

Preferably, the protein to be stored is a peptide fragment. Preferably, the peptide fragment has been obtained by the enzymatic hydrolysis of a larger protein.

The protein to be stored may be present in a suitable medium, such as a solution, gel or macerated gel plug suspension, which may then be applied to the substrate.

The following discussion pertains to the first and second aspects of the invention, unless otherwise indicated.

The substrate of the invention may, for example, be particulate or it may be in a laminar form, such as a sheet or a membrane, which may be a single layer or a multilayer structure supported or unsupported on a porous or non-porous structure and unreinforced or reinforced with a porous scrim. Alternatively, the substrate may be three-dimensional in form and may, for example, be an amorphous form.

There are a number of fibrous forms which may be present in the substrate. These include silica-based materials (e.g. glass and quartz), asbestos, metal, zirconia, alumina, carbon, ceramics, polyamides (e.g. nylon), polyesters, acrylics, polyolefins (e.g. polyethylene and polypropylene), polyimides, polyvinylchlorides, PTFE, poly-aramids (e.g. Kevlar), polysaccharides including regenerated and non-regenerated structures where the monosaccharide unit is an aldose or a ketose., such as glucose or mannose. Examples of such polysaccharides include cellulose. Preferably, the cellulose material is of microbial or plant origin. Thus, cotton, wood, straw, flax, jute, hemp and manila may be used to manufacture the substrate. Other polysaccharides include chitin and chitosan.

Preferably, the substrate comprises at least 20%, 30%, 40%, 50%, 60%, 70%, 80%, 90% or 95%, 99%, 99.5%, or 99.9% of one, two, three or four or moreof the above fibrous forms. Preferably, these percentages are percentages by weight. For the avoidance of doubt, where the substrate employed in the present invention is said to comprise a given percentage by weight of a certain constituent (in this instance one or more of the above-mentioned fibrous forms) the percentage weight of the constituent refers to the percentage by weight of that constituent in the substrate prior to treatment of the substrate with a polyhydric compound.

Preferably, the substrate comprises at least 20%, 30%, 40%, 50%, 60%, 70%, 80%, 90% or 95%, 99%, 99.5% or 99.9% by weight of one or more polysaccharides where the monosaccharide unit is an aldose or a ketose, such as glucose or mannose. As indicated above, the term polysaccharide includes both regenerated and non-regenerated structures. Preferably, the substrate comprises at least 20%, 30%, 40%, 50%, 60%, 65%, 70%, 80%, 90% or 95%, 99%, 99.5% or 99.9% by weight of cellulose. Preferably, the cellulose material is of microbial or plant origin, e.g. cotton, wood, straw, flax, jute, hemp and manila. Other polysaccharides include chitin and chitosan.

The substrate could comprise a single type of fibre; suitably the type of fibre is one of the types of fibre mentioned above, e.g. cellulose fibres. Optionally, the substrate may comprise further substances except, of course, a further fibre type. Optionally the substrate may comprise additionally a particulate material, such as for example, silica gel particles.

Preferably, the substrate consists of a single type of fibre, suitably the fibres are one of the types mentioned above, e.g. cellulose fibres. Alternatively, the substrate could comprise more than one type of fibre and the substrate may thus be a composite. For example, the substrate may be a cellulose/glass mixed furnish.

In one embodiment of the invention the substrate is a cellulose paper. Preferably, the cellulose paper is one or more of the following types of cellulose paper: a smooth surface cellulose paper, a calendered cellulose paper, an acid-treated cellulose paper, a hardened cellulose paper, an un-hardened cellulose paper, a quantitative cellulose paper, a qualitative cellulose paper and a strengthened cellulose paper.

Preferably, the substrate is one of the following papers: Whatman Grade 31ET (smooth cellulose), Whatman Grade 31ETF (smooth cellulose), Grade 50 (calendered, hardened cellulose), BFC 180 (smooth cellulose) and 3MM Chr (smooth cellulose). Preferably, the substrate is a cellulose paper which has properties substantially similar to one of these cellulose papers. Typical properties of these papers are set forth below.

31ET (Smooth Cellulose)

| | | |
|---|---|---|
| Basis Weight | 192 | g/m² |
| Spec Typical Thickness | 500 | μm @ 53 kPa |
| Gurley Air Porosity (5 oz cylinder) | 4.7 | s/100 ml/in² |
| Tensile MD Dry | 54.7 | N/15 mm |
| Tensile MD Wet | 6.4 | N/15 mm |
| Water absorption | 46 | mg/cm² |
| Klemm wicking | 2:59 | min:sec/7.5 cm |

31ETF (smooth cellulose)—as for 31ET (smooth cellulose), but 31ETF (smooth cellulose) is handled using gloves and face masks in order to minimise the risk of contamination.

Grade 50 (Calendered Hardened Cellulose)

| | | |
|---|---|---|
| Basis Weight | 97 | g/m² |
| Spec Typical Thickness | 115 | μm @ 53 kPa |
| Gurley Air Porosity (5 oz cylinder) | 96.0 | s/100 ml/in² |
| Tensile MD Dry | 84.0 | N/15 mm |
| Tensile MD Wet | 17.0 | N/15 mm |
| Water absorption | 12 | mg/cm² |
| Klemm wicking | 37:31 | min:sec/7.5 cm |

BFC 180 (Smooth Cellulose)

| | | |
|---|---|---|
| Basis Weight | 192 | g/m² |
| Spec Typical Thickness | 490 | μm @ 53 kPa |
| Gurley Air Porosity (5 oz cylinder) | 3.2 | s/100 ml/in² |
| Tensile MD Dry | 41.3 | N/15 mm |
| Tensile MD Wet | 2.0 | N/15 mm |
| Water absorption | 51 | mg/cm² |
| Klemm wicking | 3:25 | min:sec/7.5 cm |

3MM chr (Smooth Cellulose)

| | | |
|---|---|---|
| Basis Weight | 189 | g/m² |
| Spec Typical Thickness | 335 | μm @ 53 kPa |
| Gurley Air Porosity (5 oz cylinder) | 19.1 | s/100 ml/in² |
| Tensile MD Dry | 86.8 | N/15 mm |
| Tensile MD Wet | 3.8 | N/15 mm |
| Water absorption | 31 | mg/cm² |
| Klemm wicking | 10:02 | min:sec/7.5 cm |

Suitable materials for a membranous substrate include: nitrocellulose, cellulose acetate, regenerated cellulose, PVDF, polyether sulfone, polysulfone, PTFE, ceramic, silver, metal oxide, nylon, cellulose, or polypropylene, and mixtures of these materials. The membrane may be metallised. The membrane may be a track-etched membranes e.g. polycarbonate and PET. The substrate may comprise or consist of one of these materials.

Preferably, the substrate is a cellulose nitrate 5 unsupported membrane or a membrane having properties substantially similar to a cellulose nitrate 5 unsupported membrane. Typical properties of a cellulose nitrate 5 unsupported membrane paper is set forth below.

Cellulose Nitrate 5 Unsupported

Type: Membrane
Description: Cast polymeric membrane with nominal pore size
Composition: 100% Cellulose nitrate

| | | | |
|---|---|---|---|
| Nominal Pore | 5 micron | Spec Typical | 125@ 20 kPa |
| Size | | Thickness (μm) | |
| Air Flow Rate | 37 l/ | Gurley Air | 8.5 s/200 ml/in² |
| | min/cm² | Porosity | |
| Water Flow | 500 ml/ | Wicking Rate | 37 s(1.5 cm × 2 cm) |
| Rate | min/cm² | | |
| Dry Burst | 1.0 bar | Max Service | 80° C. |
| | | Temperature | |

In one embodiment of the invention it is preferred that the substrate comprises less than 95%, 90%, 80%, 70%, 60%, 50%, 40%, 35%, 30%, 20%, 10% or 5% by weight of polyester and/or polyamide fibres. In one embodiment the substrate does not comprise polyester and/or polyamide fibres. In one embodiment of the invention the substrate is a melt blown polypropylene filter medium.

Suitably, the substrate is water insoluble and maintains its structural integrity when exposed to one or more of the following: an aqueous solution, an organic solution, a polar solution, a non-polar solution, a biological fluid such as whole blood or serum.

Preferably, the substrate comprises less than 95%, 90%, 80%, 70%, 60%, 50%, 40%, 30%, 20%, or 10% by weight of glass. Preferably, the substrate comprises less than 95%, 90%, 80%, 70%, 60 %, 50%, 40%, 30%, 20%, or 10% by weight of glass or quartz. Preferably, the substrate comprises less than 95%, 90%, 80%, 70%, 60%, 50%, 40%, 30%, 20%, or 10% by weight of one or more oxides of silicon. In one embodiment the substrate does not comprise glass. Preferably, the substrate does not comprise glass or quartz. Preferably the substrate does not comprise an oxide of silicon.

The term “polyhydric compound” is used herein to refer to a compound which comprises at least one hydroxyl group linked to an inorganic or organic structure including aliphatic and/or aromatic groups in a linear and/or cyclic form. The polyhydric compound is preferably water soluble although water insoluble forms, such as a fibrous PVA, may also be used

Preferably, the polyhydric compound is selected from the group consisting of: a PVA having a molecular weight of 9000-186000 and a degree of hydrolysis of at least 70%, preferably 80%; glycerol; sucrose; carrageenan; xanthan gum and pectin. Preferably, the polyhydric compound is a polymeric compound.

Preferably, the polyhydric compound is a PVA having a degree of hydrolysis of greater than 70%, most preferably between 80% and 99%, 82% and 95%, 84% and 92%, or between 86.5% and 89%. Preferably, the polyhydric compound is a PVA having a degree of hydrolysis less than about 97.5%, 97.0%, 96.5%, or 96.0%. Preferably, the polyhydric compound is a PVA having a degree of hydrolysis less than about 97.0%.

Preferably, the PVA has a molecular weight between 15,000 and 19,000, 16,000 and 18,000, about 15,000, 16,500 and 17,500, 17,100 and 17,300. Preferably, the PVA has a molecular weight of about 17,200.

Preferably, a PVA having a molecular weight of about 17,200 and which is 86.5-89% hydrolysed is used. Such a PVA is manufactured by Nippon Gohsei under the trade name GL-03.

Preferably, the polyhydric compound is dissolved in water or a suitable buffer (eg a Tris/HCl buffer, or a Tris/EDTA buffer).

With regard to the first aspect of the invention, where the protein to be stored is applied to the substrate as a solution of the protein, the solution of the protein to be stored comprises the protein in a suitable solvent which is compatible with the stabilisation of the protein, e.g. an aqueous or organic solvent or mixtures thereof, the solvent may be polar or non-polar.

Preferably, the protein solution comprises, in addition to the protein to be stabilised, one or more further substances. For example, the protein solution may comprise a substance for maintaining the activity or avoiding the deactivation of the protein in solution, for example a buffer-substance for the maintenance of a desired pH, detergents, salts or particular protective substances such as albumin or saccharose. The protein solution may also comprise a reducing agent which may, for example, be beneficial in helping to retain the activity of a protein bearing one or more sulfhydryl groups. It will be appreciated that where the protein to be stored is present in some other medium, e.g. a gel, the substances mentioned in this paragraph may be present in the gel.

In an alternative embodiment, the substrate may be treated with one of the substances mentioned in the above paragraph prior to, or subsequent to, the application of the protein to the substrate.

Similarly, with regard to the second aspect of the invention, where the protein to be stored is applied to the substrate as a solution of the protein, the solution of the protein to be stored comprises the protein in a suitable solvent which is compatible with the chemical stabilisation of the protein, e.g. an aqueous or organic solvent or mixtures thereof, the solvent may be polar or non-polar.

Preferably, the protein is an enzyme. Preferably, the protein is one used for enzymatic determinations, such as enzymes, or one used for immunological detection reactions, such as an antigen, an antibody (monoclonal or polyclonal) or a fragment thereof, or a conjugate of an immunologically active substance with a labelling substance, for example an enzyme.

Preferably, the protein is soluble in water. It should be noted that the term “solution” as used herein includes suspensions of proteins.

In one embodiment of the first and second aspects of the invention, one or more blood proteins is stored. Preferably, one or more of the following proteins is stored: plasminogen, serotransferrin, albumin, A-1 antitrypsin, A-1 antichymotrypsin, one or more Ig heavy chains, haptoglobin, one or more Ig light chains, Apo A-1 and/or one or more complement proteins. Such proteins may be obtained from blood or may be recombinantly produced.

In one embodiment of the first and second aspects of the invention, the proteome of whole blood or of a blood fraction is stored, for example the proteome of plasma or serum is stored. Accordingly, the various proteins of the proteome may be stabilised by the polyhydric treated substrate.

One embodiment of the invention provides a proteome stored on a substrate which has been treated with a polyhydric compound and dried, wherein the amount of the polyhydric compound present in the substrate is sufficient to stabilise the activity of the proteins of the proteome, and wherein the substrate does not consist of glass. It will be appreciated that the proteome may be stored on the substrate in accordance with the methods of the first and second aspsects of the invention. Preferably, the proteome of whole blood or of a blood fraction is stored, for example the proteome of plasma or serum is stored.

Another embodiment of the invention provides a proteome stored on a substrate which has been treated with a polyhydric compound and dried, wherein the amount of the polyhydric compound present in the substrate is sufficient to chemically stabilise the proteins of the proteome, and wherein the substrate does not consist of glass. It will be appreciated that the proteome may be stored on the substrate in accordance with the methods of the first and second aspsects of the invention. Preferably, the proteome of whole blood or of a blood fraction is stored, for example the proteome of plasma or serum is stored.

In one embodiment of the invention, two or more proteins may be simultaneously stored and stabilised on the substrate. For instance, two or more solutions each comprising a different protein to be stably stored could be applied to the substrate. Alternatively, a solution (or a gel, macerated gel plug suspension etc.) comprising two or more proteins to be stably stored may be applied to the substrate.

Preferably, the substrate is treated with a solution of a polyhydric compound of 0.001% to 10% (w/w), 0.01% to 10% (w/w), 0.1% to 10% (w/w), 2% to 5% (w/w), or 2.5% and 3.5% (w/w). Preferably, the substrate is treated with a PVA solution of about 2% or about 3% (w/w). Preferably the polyhydric compound is a PVA.

Preferably, the substrate is a paper having the properties of Whatman Grade 31ET paper (smooth cellulose) or 31ETF paper (smooth cellulose) and it is coated with 3% (w/w) solution of PVA grade GL-03 (Nippon Gohsei) Mol Wt 17200, 86.5-89% hydrolysed. For 31ET paper, this gives a sheet containing ˜1.8% (w/w) PVA.

In another embodiment, it is preferred that the substrate is a paper having the properties of Whatman Grade 31ET paper (smooth cellulose) or 31ETF paper (smooth cellulose) and it is coated with 1.75% to 2.25% (w/w) solution of PVA grade GL-03 (Nippon Gohsei) Mol Wt 17200, 86.5-89% hydrolysed, preferably 2% (w/w) solution of PVA grade GL-03 (Nippon Gohsei) Mol Wt 17200, 86.5-89% hydrolysed.

Preferably, the polyhydric-treated substrate comprises a polyhydric compound at a loading of at least 0.25%, 0.5%, 1%. 1.5%, 1.8% or 2% (w/w). In the case of PVA, it is preferred that the substrate contains less than 2% (w/w) PVA, preferably about 1.8% (w/w) PVA.

It will be appreciated that if, for example, the protein is stored on a strip of cellulose paper it might be desirable to store the protein on only a portion of the strip. Accordingly, only the protein-storing region of the strip need comprise the polyhydric compound. Thus, with regard to the first aspect of the invention, if a discrete region of the strip etc. is to be used for protein storage, then a sufficient amount of the polyhydric compound to stabilise the protein for the desired time (e.g. for at least three weeks), need be present only in this region. Similarly, with regard to the second aspect of the invention, if a discrete region of the strip etc. is to be used for protein storage, then a sufficient amount of the polyhydric compound to chemically stabilise the protein for the desired time (e.g. for at least three weeks) need be present only in this region. As noted above, it is preferred that the polyhydric compound is present at a loading of at least 0.25%, 0.5%, 1%. 1.5%, 1.8% or 2% (w/w).

The substrate may be treated with at least two of the aforementioned polyhydric compounds. With regard to the first aspect of the invention, the two or more polyhydric compounds may act synergistically together to stabilise the activity of the protein. With regard to the second aspect of the invention, the two or more polyhydric compounds may act synergistically together to chemically stabilise the protein.

By a “substrate which has been treated with a polyhydric compound and dried” is meant a substrate from which excess moisture has been removed. It will be appreciated from the discussion below that the substrate should retain some residual moisture so as not to impair the stabilising abilities of the substrate. As noted below, there is sufficient water present in ambient stored paper (˜4% w/w) to provide a stabilising environment provided that hydrophilic materials such as PVA are present.

Preferably, the protein to be stored has been obtained from a proteomic sample (e.g. from a plasma or serum proteome).

Preferably, the protein to be stored has been subjected to chromatographic purification. Preferably, the effluent stream from the chromatographic system is directly applied to the substrate.

Alternatively, a proteomic sample is digested with an enzyme, such as trypsin and the digested protein fragments are subjected to chromatographic purification. In this alternative preferred embodiment, the effluent stream from the chromatographic system is directly applied to the substrate.

As mentioned above, the study of protein function is gaining importance with the emergence of proteomics. In most applications the proteome is fractionated using 2-dimensional electrophoresis. This technique though widely used is denaturing and so functional determination of proteins is by implication rather than demonstration. In order to obtain functionally active proteins chromatographic techniques are preferred. These are well established for the large-scale purification of proteins. In the field of proteomics these separations are scaled down to Microsystems such as capillary electrophoresis and capillary HPLC. While the eluate may be directly introduced to a mass spectrometer, typically by electrospray, there may be a requirement to collect fractions and/or split the streams. In either case the volumes of the fractions will be very small, eg 10's or 100's of nanolitres.

In order to effect fraction collection from these microchromatographic techniques the protein storage media described herein may be used. Briefly the effluent stream from the chromatographic system would be directly applied to a polyhydric-treated substrate in accordance with the first or second aspect of the invention. This would allow for the stable storage of protein fractions, for archiving and/or subsequent analysis and characterisation.

Thus, in one embodiment of the first or second aspect of the invention, the protein to be stored has been obtained from a proteomic sample and the protein to be stored has been subjected to chromatographic purification and, the effluent stream from the chromatographic system containing the protein to be stored is directly applied to the substrate.

Where the protein has been subjected to chromatographic purification, it is preferred that the substrate may be in the form of a moving reel or strip. Preferably, the substrate is in the form of a moving reel or strip such that the protein is applied in discrete sequential places, or could be held in a static device such as a multiwell plate and the effluent stream directed to individual wells or regions by a moveable head such as are used in liquid handling and dispensing systems.

We have demonstrated that various non-glass substrates enable stable storage of purified protein samples of various origin and type for four weeks or more at ambient temperatures in an uncontrolled environment with retention of significant biological activity. Whilst not wishing to be bound by any particular theory, it is believed that water molecules are essential for stabilising proteins during drying for subsequent storage. There is sufficient water present in ambient stored paper (˜4% w/w) to provide a stabilising environment provided that hydrophilic materials such as PVA are present. It is believed that the mode of action of the polyhydric compounds, is to provide a hydrating region that enables the retention of water molecules within the protein molecule such that the native secondary, tertiary and quaternary structures are retained on drying and storage, thereby maintaining biological activity.

Following storage, the protein can be eluted from the substrate for subsequent use. This might include, for example, electrophoretic analysis or MALDI-TOF MS for proteome analysis. Alternatively, the eluted protein may be used in an analytical method and may thus, for example, be used to assay liquids such as biological fluids. In the case where the eluted protein is a proteinase, the eluted proteinase solution may be used to digest protein. Thus, for example, trypsin may be stably stored in accordance with the invention, subsequently eluted from the storage substrate and then used to digest a protein.

Following storage, the protein may be used in an analytical assay (e.g. to assay a biological fluid) and may thus, for example, be used to assay liquids such as biological fluids. The protein may still be in combination with the paper when the analytical assay is undertaken or alternatively the protein may be eluted from the substrate and the eluted protein then used in the assay. The assay may be performed on a living human or animal body. In one embodiment, it is preferred that the assay is not performed on a living human or animal body. In one embodiment, the assay is performed on a biological sample, e.g. fluid, obtained from a human or animal body.

Accordingly, a third aspect of the present invention provides an analytical assay performed using a protein which has been stably-stored by a method of the first or second aspect of the invention. The assay may be performed on a living human or animal body. In one embodiment, it is preferred that the assay is not performed on a living human or animal body. In one embodiment, the assay is performed on a biological sample, e.g. fluid, obtained from a human or animal body.

The digestion of proteins by trypsin (and other proteinases) plays an important role in proteomics. Accordingly, in one embodiment of the first aspect of the invention the protein to be stored is a proteinase.

Preferably the proteinase is a serine proteinase, for example trypsin, chymotrypsin or kallikrein, suitably bovine pancreatic trypsin. Other proteinases that may be useful might include Proteinase K, lysosomal enzymes, cathepsins (e.g. cathepsin B, C, D, G, H or N), papain and pepsin.

Preferably, the proteinase is an endopeptidase. Preferably the endopeptidase is bromelain, cathepsin B, cathepsin D, cathepsin G, chymotrypsin, clostripain, collagenase, dispase, endoproteinase Arg-C, endoproteinase Asp-N, endoproteinase Glu-C, endoproteinase Lys-C, Factor Xa, Kallikrein, Papain, Pepsin, Plasmin, Proteinase K, Subtilisin, thermolysin, thrombin or trypsin.

Preferably, the proteinase is an exopeptidase. Preferably, the exopeptidase is acylamino-acid-releasing enzyme, aminopeptidase M, carboxypeptidase A, carboxypeptidase B, carboxypeptidase P, carboxypeptidase Y, cathepsin C, leucine aminopeptidase or pyroglutamate alminopeptidase.

Further proteinases will be known to those skilled in the art.

In order to effect a superior digest the present invention provides for the preparation and application of a proteinase-loaded substrate. Such a material would have application to, for example, crude proteomes, enriched proteomes and other protein samples requiring tryptic digestion. The proteinase may be eluted from the substrate prior to use or the protein to be digested may be added to the proteinase-loaded substrate.

Briefly, a solution of the proteinase could be applied to a substrate as described in the first aspect of the invention. This will provide a proteinase-loaded substrate where the proteinase is stably stored. The amount of the proteinase required would be known to those skilled in the art, or could readily be determined by those skilled in the art. With regard to trypsin, typically up to 250 ng is used per gel plug (Butt, A. et al. Proteomics (2001) 1 42-53-125-250 ng; Devreese, B et al. Rapid Commun. Mass Spectrom. (2001) 15 50-56-125 ng; Sickmann et al. Electrophoresis (2001) 22 1669-1676 -100 ng).

Protein, e.g. in the form of a gel plug, macerated gel plug suspension or electroeluted components from a region of a gel, can then be applied to the surface of the proteinase-loaded substrate. This would facilitate in situ digestion of the protein under suitable digestion conditions. Suitable digestion conditions will be well known to those skilled in the art or could be readily determined by those skilled in the art. Suitable conditions for tryptic digest include incubation of the protein at pH˜8.0 at 37° C. overnight.

Optionally, one or more subsequent protein samples could be applied to the substrate which would enable a build up of a concentrated zone of digested protein to be obtained for subsequent analysis.

As indicated above, it is also envisaged the proteinase may be eluted from the substrate and then used to effect protein digestion.

The versatility of the base material is such that other suitable enzyme systems may be used to in addition to, or instead of, trypsin to effect the appropriate in situ biotransformation.

Thus, a fourth aspect of the present invention provides a method of digesting a protein, the method comprising:
(i) stably storing a proteinase on a substrate according to the method of the first aspect of the invention to thereby obtain a proteinase-loaded substrate;
(ii) applying the protein to be digested to the proteinase-loaded substrate under suitable conditions for the digestion of said protein to thereby effect digestion of the protein; or eluting the proteinase from the proteinase-loaded substrate to obtain a proteinase solution which is then used to effect digestion of the protein.

Similarly, a fifth aspect of the present invention provides a method of digesting a protein, the method comprising either:
applying the protein to be digested to a proteinase-loaded substrate under suitable conditions for the digestion of said protein to thereby effect digestion of the protein 30 wherein the proteinase is stably stored on the substrate according to the method of the first aspect of the invention to thereby produce the proteinase-loaded substrate, or
using a proteinase solution comprising proteinase eluted from a proteinase-loaded substrate to effect digestion of the protein wherein the proteinase has been stably stored on the substrate according to the method of claim 23 or 24 to thereby produce the proteinase-loaded substrate,

Preferably the proteinase is a serine proteinase, for example trypsin, chymotrypsin or kallikrein, suitably bovine pancreatic trypsin. Other proteinases that may be useful might include Proteinase K, lysosomal enzymes, cathepsins (e.g. cathepsin B, C, D, G, H or N), papain and pepsin.

Preferably, the proteinase is an endopeptidase. Preferably the endopeptidase is bromelain, cathepsin B, cathepsin D, cathepsin G, chymotrypsin, clostripain, collagenase, dispase, endoproteinase Arg-C, endoproteinase Asp-N, endoproteinase Glu-C, endoproteinase Lys-C, Factor Xa, Kallikrein, Papain, Pepsin, Plasmin, Proteinase K, Subtilisin, thermolysin, thrombin or trypsin.

Preferably, the proteinase is an exopeptidase. Preferably, the exopeptidase is acylamino-acid-releasing enzyme, aminopeptidase M, carboxypeptidase A, carboxypeptidase B, carboxypeptidase P, carboxypeptidase Y, cathepsin C, leucine aminopeptidase or pyroglutamate aminopeptidase.

Further proteinases will be known to those skilled in the art.

Preferably, the protein to be digested is applied to the proteinase-loaded substrate in the form of a gel plug, macerated gel plug suspension or electroeluted components (e.g. from a region of a gel) which has been subjected to electrophoresis, preferably 2D electrophoresis.

Preferably, the protein to be digested has been subjected to chromatographic purification. Preferably, the protein to be digested has been obtained from a proteomic sample. Preferably, the effluent stream from the chromatographic system containing the protein to be digested is directly applied to the substrate.

Where the protein has been subjected to chromatographic purification, it is preferred that the substrate may be in the form of a moving reel or strip such that spots are applied in discrete sequential places, or could be held in a static device such as a multiwell plate and the effluent stream directed to individual wells or regions by a moveable head such as are used in liquid handling and dispensing systems.

Preferably, the protein to be digested is one or more blood proteins. Preferably, one or more of the following proteins is digested: plasminogen, serotransferrin, albumin, A-1 antitrypsin, A-1 antichymotrypsin, one or more Ig heavy chains, haptoglobin, one or more Ig light chains, Apo A-1 and/or one or more complement proteins. Such proteins may be obtained from blood or may be recombinantly produced.

In one embodiment of the fourth and fifth aspects of the invention, the proteome of whole blood or of a blood fraction is digested, for example the proteome of plasma or serum.

Various aspects and embodiments of the present invention will now be described in more detail by way of example. It will be appreciated that modification of detail may be made without departing from the scope of the invention.

### BRIEF DESCRIPTION OF THE FIGURES

FIG. 1 is a plot of colour intensity against time for a protein stabilised on 31ET paper (smooth cellulose) treated with PVA.

FIG. 2 represents the stability of trypsin on 31ET paper (smooth cellulose) with and without PVA treatment.

FIG. 3 represents the stability of trypsin on 31ET paper (smooth cellulose) treated with PVA compared to the activity of a trypsin solution.

FIG. 4 shows the various plasma proteins. A denotes plasminogen, B serotransferrin, C albumin, D A-1 antitrypsin, E A-1 antichymotrypsin, F Ig heavy chains, G haptoglobin, H Ig light chains, I Apo A-1. J denotes the area in FIG. 9.

FIG. 5 represents the plasma proteome at time zero.

FIG. 6 represents the plasma proteome after 14 days at −80° C.

FIG. 7 represents the plasma proteome after 0 and 14 days using PVA-treated 31ETF paper (smooth cellulose).

FIG. 8 represents the plasma proteome after 0 and 14 days using non-PVA-treated 31ETF paper (smooth cellulose).

FIG. 9 illustrates the altered group of spots seen after 14 days with non-PVA-treated 31ETF paper (smooth cellulose).

FIG. 10 represents the stability of alkaline phosphatase conjugate (10 μl of 50 μg/ml) on 31ET paper (smooth cellulose) with and without PVA treatment at 20-25° C.

FIG. 11 represents the stability of 500 ng bovine pancreatic trypsin (10 μl of 50 μg/ml) on 31ET paper (smooth cellulose) with and without PVA treatment at 20-25° C.

FIG. 12: MALDI-TOF spectrum for spot treated with bovine pancreatic trypsin freshly made up in ammonium bicarbonate buffer.

FIG. 13: MALDI-TOF spectrum for spot to which trypsin on PVA-treated paper and ammonium bicarbonate buffer has been added.

FIG. 14: MALDI-TOF spectrum for spot treated with bovine pancreatic trypsin freshly made up in ammonium bicarbonate buffer.

FIG. 15: MALDI-TOF spectrum for spot to which trypsin on PVA-treated paper and ammonium bicarbonate buffer has been added.

FIG. 16: spectrum for spot treated with bovine pancreatic trypsin freshly made up in ammonium bicarbonate buffer.

FIG. 17: MALDI-TOF spectrum for spot to which trypsin on PVA-treated paper and ammonium bicarbonate buffer has been added.

FIG. 18: spectrum for spot treated with bovine pancreatic trypsin freshly made up in ammonium bicarbonate buffer.

FIG. 19: MALDI-TOF spectrum for spot to which trypsin on PVA-treated paper and ammonium bicarbonate buffer has been added.

FIG. 20: ELISA at day 0 comparing the signal intensity obtained when antibody is stored on PVA-treated paper (“Protein Saver”) and when stored on a control substrate.

FIG. 21: ELISA at day 28 comparing the signal intensity obtained when antibody is stored on PVA-treated paper (“Protein Saver”) and when stored on a control substrate.

FIG. 22: SDS-PAGE at day 28. 1: Reduced; Control; 2: Non-Reduced; Control; 3: Reduced; Protein Saver; 4: Non-Reduced; Protein Saver; 5: Reduced Non-Spotted; −20° C.; 6: Non- Reduced Non-Spotted; −20° C.; 7: Reduced Non-Spotted; 4° C.; 8: Non-Reduced Non-Spotted; 4° C.; 9: Reduced Non-Spotted; 20-25° C.; 10: Non-Reduced Non-Spotted; 20-25° C.; 11: Marker.

FIG. 23: ELISA at day 0 comparing the signal intensity obtained when tissue culture supernatant is stored on PVA-treated paper (“Protein Saver”) and when stored on a control substrate.

FIG. 24: ELISA at day 28 comparing the signal intensity obtained when tissue culture supernatant is stored on PVA-treated paper (“Protein Saver”) and when stored on a control substrate.

FIG. 25: SDS-PAGE at day 28. 1: Reduced; Control; 2: Non-Reduced; Control; 3: Reduced; Protein Saver; 4: Non-Reduced; Protein Saver; 5: Reduced Non-Spotted; −20° C.; 6: Non-Reduced Non-Spotted; −-20° C.; 7: Reduced Non-Spotted; 4° C.; 8: Non-Reduced Non-Spotted; 4° C.; 9: Reduced Non-Spotted; 20-25° C.; 10: Non-Reduced Non-Spotted; 20-25° C.; 11: Marker.

FIG. 26: Mass spectrum of a fresh tryptic digest of BSA.

FIG. 27: Mass spectrum of tryptic digests of BSA stored at 4° C.

FIG. 28: Mass spectrum of tryptic digests of BSA stored at 20° C.

FIG. 29: Mass spectrum of tryptic digests of BSA stored on PVA-treated paper at 20° C.

FIG. 30: Mass spectrum of tryptic digests of BSA stored on Control substrate at 20° C.

### EXAMPLES

### Example 1

The following substrates were used: cellulose papers Whatman Grade 31ET (smooth cellulose), Grade 50 (calendered, hardened cellulose), BFC 180 (smooth cellulose), 3MM Chr (smooth cellulose); a nitrocellulose 5 micron unsupported membrane and a-melt blown polypropylene filter medium.

It has been demonstrated that the following polyhydric compounds may be used to treat the above substrates. Water soluble polyvinyl alcohols ranging in molecular weight from 9000-186000 and degrees of hydrolysis ranging from 80-99+%, glycerol, sucrose, carrageenan, xanthan gum and pectin. In addition a fibrous PVA (VPB 101, 70-80000 MW) was included in a cellulose paper structure at a loading of 1.8% (w/w). We also describe a diluted solution of hen egg white as a potential coating solution containing a complex mixture of biological components.

The following protein systems were used to test selected substrates. Protein A-alkaline phosphatase conjugate (PAAP) (assay for alkaline phosphatase activity on the surface of the sheet), alkaline phosphatase (assay for alkaline phosphatase activity on the surface of the sheet), soybean trypsin inhibitor (assay for residual bovine pancreatic trypsin activity in solution following incubation of SBTI discs with a trypsin solution), bovine pancreatic trypsin (assay for trypsin activity in solution following incubation of trypsin discs with 0.1M Tris/HCl buffer, pH 8.0, containing 0.1M NaCl for 15 min and removal of the disc of substrate).

Briefly, a 0.1 mg/ml solution of PAAP or a 0.2 mg/ml solution of alkaine phosphatase was prepared in PBS (1.00M sodium phosphate buffer, pH 7.4 containing 0.137M NaCl and 0.0027M KCI). A 5 μg/ml solution of trypsin was prepared in 0.137M NaCl and a 1 mg/ml solution of soybean trypsin inhibitor was prepared in 0.1M sodium phosphate buffer, pH 6.5 containing 0.1M NaCl. Aliquots of protein solutions were applied to defined regions of each substrate with an untreated substrate sample as control. PAAP and alkaline phosphatase used 10 μl, trypsin used 20 μl and soybean trypsin inhibitor used 10 μl. The papers were allowed to air dry and sheets were stored under ambient conditions at room temperature.

Polyhydric compounds were dissolved in water, 0.025M Tris/HCl buffer, pH 7.5 or 0.025M Tris/EDTA buffer, pH 7.5.

Coating solutions were prepared by dissolving the polyhydric compounds to prescribed concentrations and/or the maximum concentrations to give a free-flowing liquid suitable for dipping the substrate in. While this is dependent on the polyhydric compound, the maximum concentration of any compound tested was 20% (w/w). A typical coating method is as follows. Dip a sheet of substrate in the coating solution (eg 500 ml) and gently agitate for about 10 sec. Remove the sheet and allow excess coating solution to drain away. Remove residual coating solution from the surface by blotting with an absorbent cellulose paper. Dry the sheet using a heated cylinder at 90-100° C. for about 2 min.

The levels of protein activity remaining after storage were assayed using colorimetric assays. Scanning of the spot of protein and digitising the colour intensity indicate that activity losses are insignificant. Examples of such data obtained for PAAP conjugate are tabulated below.

| | 3% PVA treated | | Untreated | |
|---|---|---|---|---|
| Substrate | Day 0 | 4 weeks | Day 0 | 4 weeks |
| 31ET (smooth | 218 | 217 | 229 | 244 |
| cellulose) | | | | |
| BFC180 | 212 | 214 | 222 | 240 (2 weeks) |
| 3MM Chr | 214 | 212 | 222 | 242 |
| Grade 50 | 218 | 203 | Not determined | Not determined |
| MB Polypro. | 164 | 149 | Not determined | Not determined |
| 31ET (smooth | 209 | 218 | Not determined | Not determined |
| cellulose) - egg | | | | |
| white | | | | |

In this study the higher numerical score the lighter the spot with a white spot indicating no detectable activity as having a value of ˜250. Actual values at Day 0 depend on the related wicking rates of each grade of paper since this affects spot diameter and hence concentration of active enzyme.

In all cases tested, stored proteins retained biological activity following storage under ambient conditions at room temperature for at least four weeks. Substrates that were not treated with a polyhdric compound rapidly lose protein activity with significant losses between 3 and 7 days.

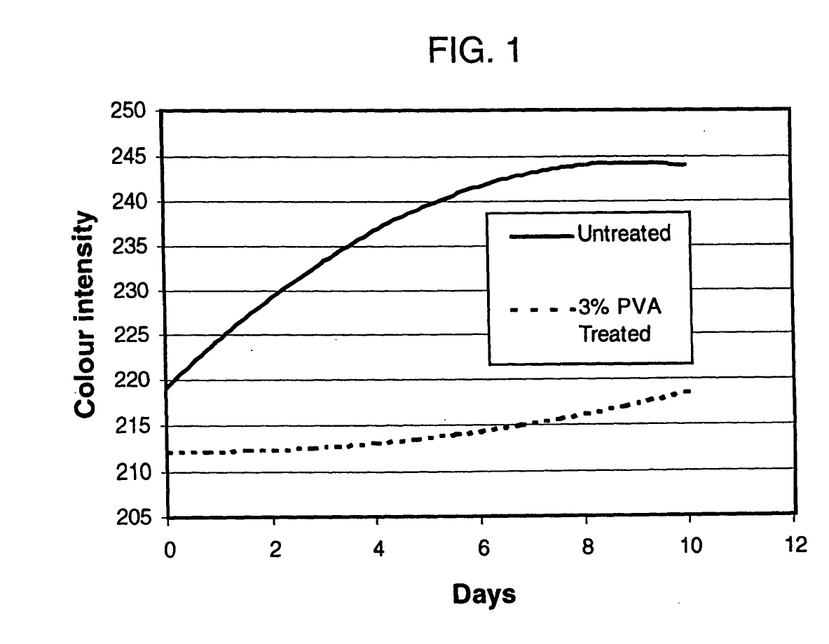

In a similar experiment,31ET (smooth cellulose)/PVA sheets were produced as described above and the activity of the PAAP conjugate measured over the first ten days of storage. The resulting data are shown below and in FIG. 1.

| | |
|---|---|
| Time | Colour Intensity |

| (days) | Untreated | Treated |
|---|---|---|
| 0 | 215 | 211 |
| 1 | 229 | 214 |
| 2 | 231 | 211 |
| 3 | 232 | 214 |
| 7 | 241 | 214 |
| 10 | 245 | 219 |

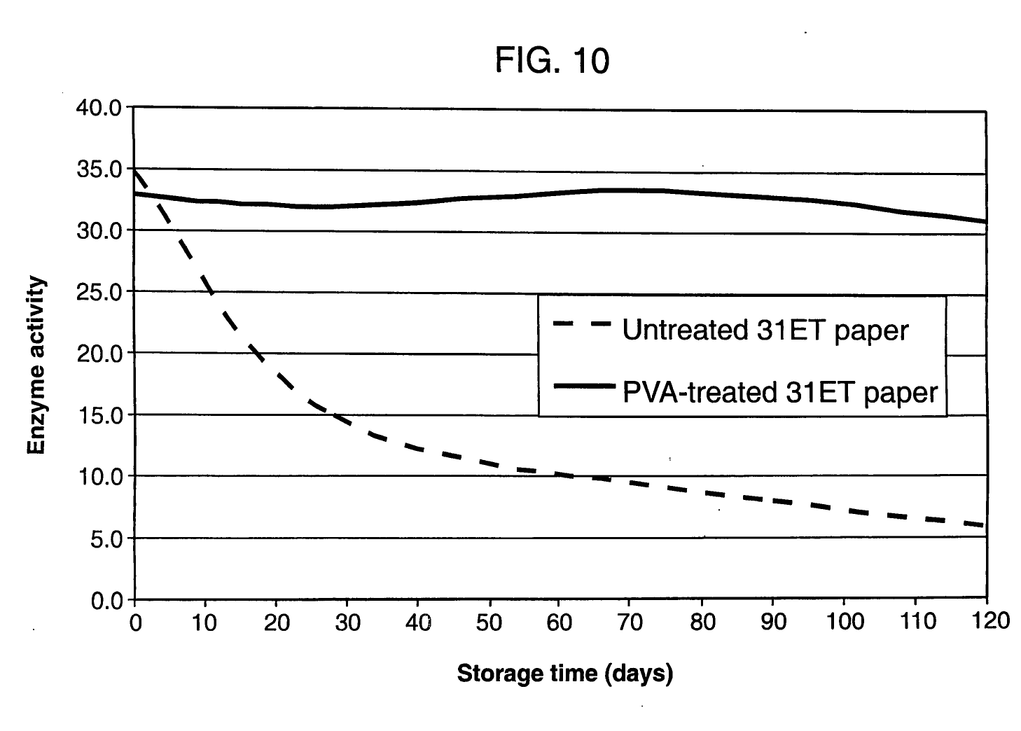

In addition to the above data, an ongoing study with 31ET (smooth cellulose)/PVA sheets indicates that biological activity is retained for at least 120 days (FIG. 10). More recently, further data has been obtained which indicates that no significant reduction in biological activity occurs even after 12 months.

Further data has been obtained on the storage of Alkaline Phosphatase on Nitrocellulose and PET-backed Nitrocellulose hand dipped in PVA (GL-03, 3% w/w). Samples were stored at ambient conditions (in the lab draw) for 182 days. No deterioration in activity was observed in the PVA coated samples but activity for the control samples have now all but completely gone.

### Example 2

Trypsin Spotting and Assays

Materials and Methods

Preparation of Trypsin Solution.

Bovine pancreatic trypsin (Sigma T8003, lot no. 62H8090) containing ˜10600 BAEe Units/mg solid was dissolved in demineralised water to give a 1 mg/ml solution. 1 ml of this solution was diluted to 200 ml with 100 mM Tris/HCl buffer, pH8.0 containing 100 mM NaCl to give a 5 μg/ml working solution containing ˜53 Units/ml. This solution was used throughout for spotting samples.

Sample Spotting

Each paper sample square (approx. 23×19 mm) was spotted with 10 μl of the 5 μg/ml trypsin solution, using an autopipette, and the spots allowed to dry under ambient conditions (approx. 10 minutes). One square per group of four is not spotted and is used as a blank control. Each spot contains 50 ng trypsin and ˜0.53 Units activity.

On-sheet Assay of Tryspin Activity

Three spotted squares and one unspotted square were assayed simultaneously. The esterase assay was based on the method of Green G. D. J. & Shaw, E. Anal. Biochem (1979) 93 223-5 226, with slight modifications. To each square was added 10 μl of 6.33 mg/ml N-α-Benzyloxycarbonyl -L-lysinethiobenzyl ester hydrochloride (Cbz-Lys-SBzl) (Sigma C-6347) dissolved in demineralised water. Immediately after addition of the substrate was added 10 μl of 2 mg/ml 5,5′-dithiobis(2-nitrobenzoic acid) (DTNIB) (Sigma D-8130) prepared in 100 mM Tris/HCl buffer, pH8.0 containing 100 mM NaCl. The yellow spot colour is allowed to develop under ambient conditions, typically requiring 15-20 minutes incubation.

Spots may be scanned by computer and the colour image converted to a greyscale. The relative colour (grey) intensity of the different spots may then be quantified and compared to controls and background values.

Trypsin Assay In Solution.

Three spotted squares and one unspotted square were assayed simultaneously. Each individual square is cut from the sheet, corrugated slightly and placed in a 30 ml universal tube. 100 mM Tris/HCl buffer, pH8.0 containing 100 mM NaCl (4.0 ml) is added to each tube using an autopipette, a cap fitted and the tube mixed for 15 minutes at room temperature. 3 ml of the extracted solution is transferred to a bijou tube using an autopipette. To this is added 20 μl of DTNB (2 mg/ml in buffer) followed by 20 μl of Cbz-Lys-Szl (6.33mg/ml in demineralised water) and the solution roller mixed. Precisely 10 minutes after addition of the Cbz-Lys-SBzl the absorbance of the solution is measured at 412 nm against a buffer control.

A trypsin control was run by incubating 10 μl of the working trypsin solution (5 μg/ml) with 100 mM Tris/HCl buffer, pH8.0 containing 100mM NaCl (4 ml) for 15 min. Trypsin activity of 3 ml of this solution was determined as above.

Results

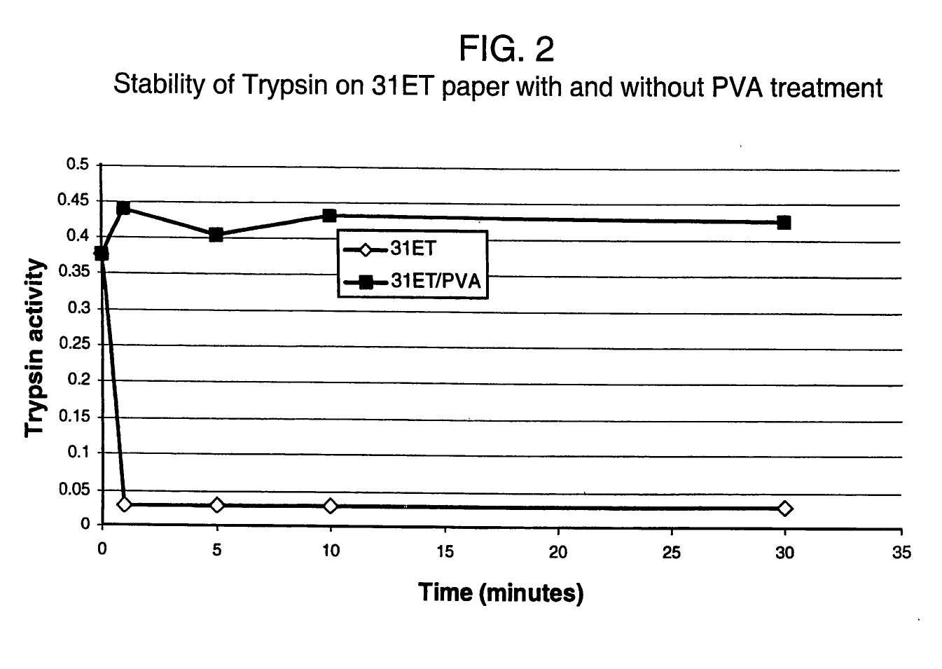

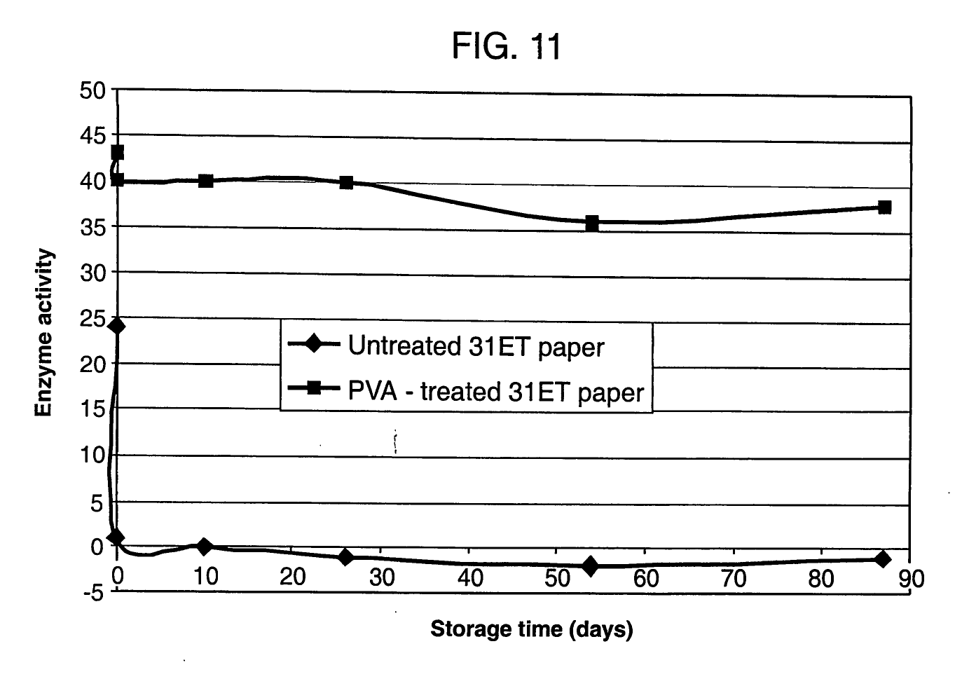

1. Bovine pancreatic trypsin (50 ng; 10 μl of 5 μ/ml) spotted, air dried and stored under ambient conditions on 31ET (smooth cellulose) papers that had been pretreated with 3% (w/v) polyvinylalcohol (PVA;GLO3) in water retained >90% enzymatic activity after 30 mins. Activity was demonstrated both using the on-sheet assay and the trypsin in solution assay. Trypsin (50 ng) spotted onto untreated 31 ET papers lost >90% of the enzymatic activity within 60 seconds of application to the paper. This activity loss was demonstrated both using the on-sheet assay and the trypsin in solution assay. These data are presented in FIG. 2 and represent trypsin activity that had been extracted from the paper using the trypsin in solution assay.
2. Trypsin (500 ng; 10 μl of 50 μg/ml) spotted, air dried and stored under ambient conditions on 31ET (smooth cellulose) papers that had been pre-treated with 3% (w/v) polyvinylalcohol (PVA;GLO3) in water retained >90% enzymatic activity after 10 mins. Similarly, data indicated that even after 87 days >90% enzymatic activity was retained (FIG. 11). Moreover, as part of an ongoing study, further data has been obtained which indicates that no significant reduction in biological activity occurs even after at least six months.

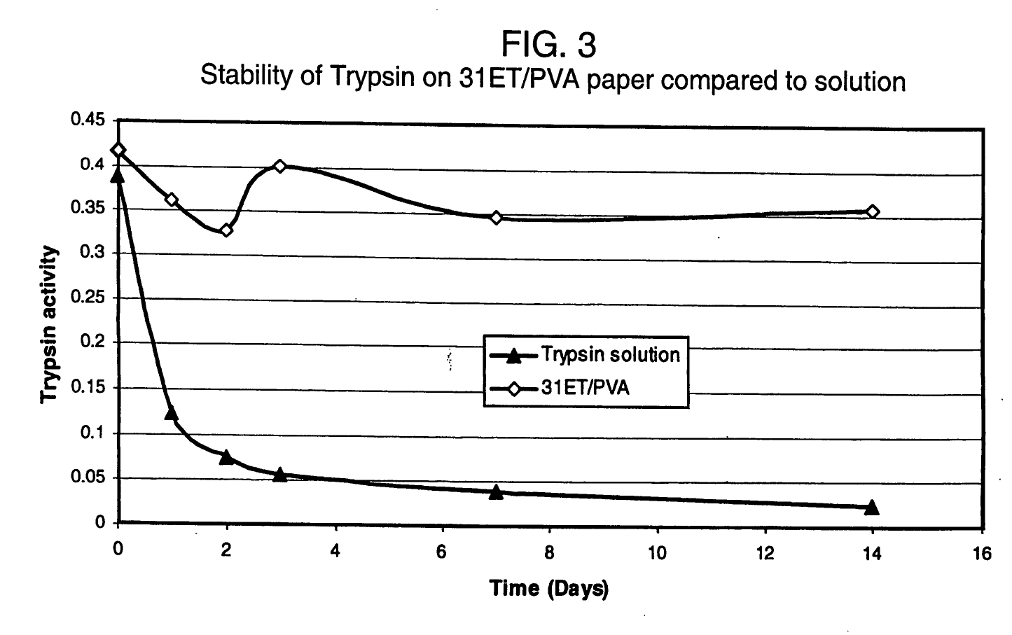

In contrast, trypsin (500 ng) spotted onto untreated 31 ET papers lost >90% of the enzymatic activity within 10 minutes of application to the paper and significant losses were observed after 4 minutes. Our results appear to suggest that applying trypsin instantaneously deactivates upon contact with 31 ET paper (and various other substrates that we have tested, with the exception of polypropylene), not even as a result of drying. . However, when the substrate has been treated with PVA significant activity is retained.
3. Trypsin solution (5 μg/ml) gradually lost activity during storage at room temperature 20-25° C. over a period of 3 days such that ˜20% activity remained after this period. Trypsin (50 ng; 10 μl of 5 μg/ml) spotted, air dried and stored under ambient conditions on 31ET papers (smooth cellulose) that had been pre-treated with 3% (w/v) polyvinylalcohol (PVA;GLO3) in water retained >90% enzymatic activity after 14 days. Data are presented in FIG. 3 for the 31ET (smooth cellulose)/PVA sample which represents trypsin activity that had been extracted from the paper using the trypsin in solution assay.
4. 3% PVA treatment on the cellulosic papers 31ET (smooth cellulose), 3MM, BFC 180 and grade 50, the glass paper F609-06, a melt-blown polypropylene and a nitrocellulose membrane all provided a storage medium for trypsin. Trypsin activity was significantly reduced on non-PVA treated substrates within the 10 min drying period whereas significant activity was retained on the PVA-treated substrates.
5. 31ET paper (smooth cellulose) treated with 3% (w/v) solutions of various PVA'ss ranging in molecular weight from 9000 -186000 and degrees of hydrolysis ranging from 80-99+% all provided a storage medium for trypsin. Significant trypsin activity was retained on the PVA-treated substrates following 30 min storage. It was surprising that there appears to be a relationship between the degree of hydrolysis of the PVA and maintenance of trypsin activity. The more hydrolysed the PVA, the less stabilisation afforded by the substrate. We have observed a similar phenomenon with other proteins although not as striking or quick as with trypsin.
6. Trypsin activity could be measured either on the surface of the PVA-treated 31ET sheets or in an eluted component following 15 min incubation of the spotted sheet in 100 mM Tris/HCl buffer pH 8.0 containing 100 mM NaCl.

### Example 3

This Example relates to the proteomic analysis of human plasma proteome stabilisation. The aim of this study was to compare the proteome of human plasma that had been stored on PVA-treated 31ETF paper (smooth cellulose) and on non-PVA treated 31ETF paper (smooth cellulose), with the proteome of human plasma that was stored at −80° C. The ability of two types of 31ETF paper to stabilise the human plasma proteome at room temperature over a period of 14 days was assessed using two-dimensional polyacrylamide gels. The two types of 31ETF paper were: (i) Whatman Grade 31ETF (smooth cellulose) coated with 2% (w/w) solution of PVA grade GL-03 (Nippon Gohsei) Mol Wt 17200, 86.5-89% hydrolysed (PVA-treated 31ETF paper); and (ii) Whatman Grade 31ETF paper (smooth cellulose) (non-PVA-treated 31ETF paper). Gels of plasma at time zero and plasma stored at −80° C. were performed as comparisons.

Protein Preparation

Human plasma was thawed, kept on ice and assayed for protein concentration using the Bradford protein assay. 100 μg of plasma protein was spotted onto 2 mm discs of each of the two types of 31ETF paper (PVA-treated and non-PVA treated). These were allowed to dry and then placed in separate bags for storage at room temperature. For time zero, 100 μg of plasma protein was added to 100 μl lysis buffer and mixed with a micro pestle for 1 minute. 350 μl of rehydration buffer was then added and this was used to rehydrate a pH 3-10 immobilised pH gradient (IPG) strip overnight.

After 14 days, each 31ETF paper disc was placed into 100 μl lysis buffer and vortexed for 1 minute. 350 μl of rehydration buffer was then added and this was used to rehydrate a 3-10 strip overnight. For the plasma stored at −-80° C., 100 μg of plasma stored at −80° C. was added to 100 μl lysis buffer and mixed with a micropestle for 1 minute. 350 μl of rehydration buffer was then added and this was used to rehydrate a pH 3-10 IPG strip overnight.

All strips were focused in the first dimension immediately after their rehydration. After focusing the strips were kept at −80° C. before separation together in the second dimension.

Two-dimensional Polyacrylamide Gel-electrophoresis

Isoelectric focusing (IEF) was performed on proteins extracted from human plasma using IPG strips (AmershamPharmacia), with pH range 3-10 (non-linear), using an in-gel rehydration method. The samples were diluted with rehydration solution prior to rehydration overnight in a reswelling tray. Total protein loads were 100 μg. After IEF the strips were equilibrated in equilibration buffer with 1% DTT for 15 minutes, followed by the same buffer with 4.8% iodoacetamide for 15 minutes, SDS-PAGE was performed using 12% T, 2.6% C. separating gels without a stacking gel using a Hoefer DALT system. The second-dimension separation was carried out overnight and was stopped as the dyefont just left the bottom of the gels. All gels were fixed and stained using the PlusOne Silver Staining (AmershamPharmacia, UK).

Results

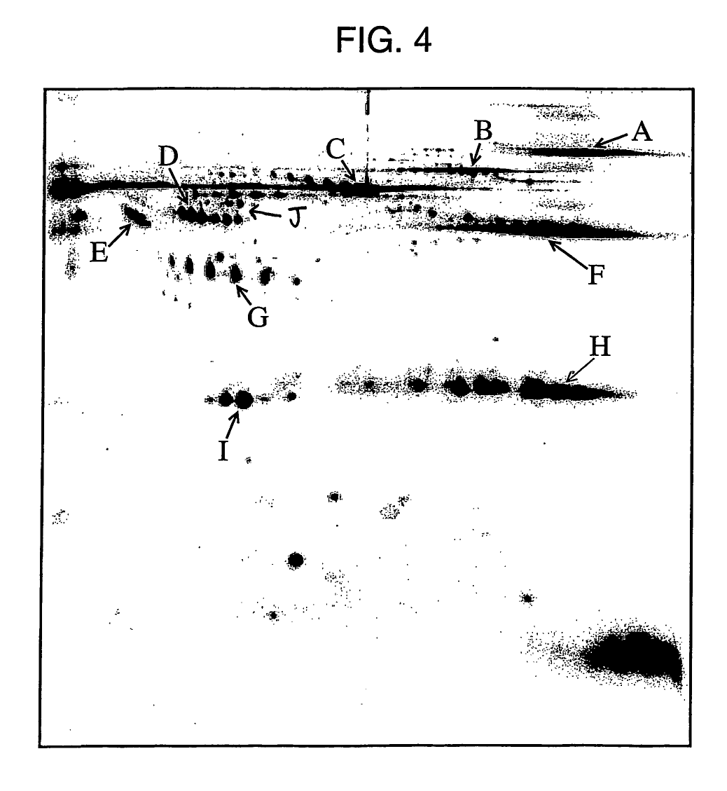

The silver stained gels are shown in FIG. 4-9. A detailed visual comparison of the spot patterns of each gel was carried out and the following observations made.

The gels were of good quality with well resolved proteins consisting of discreet spots and a number of related spots appearing in extended charge-trains. The spot patterns were consistent with those expected from human plasma. A number of known plasma proteins that appear on the gels are indicated in FIG. 4.

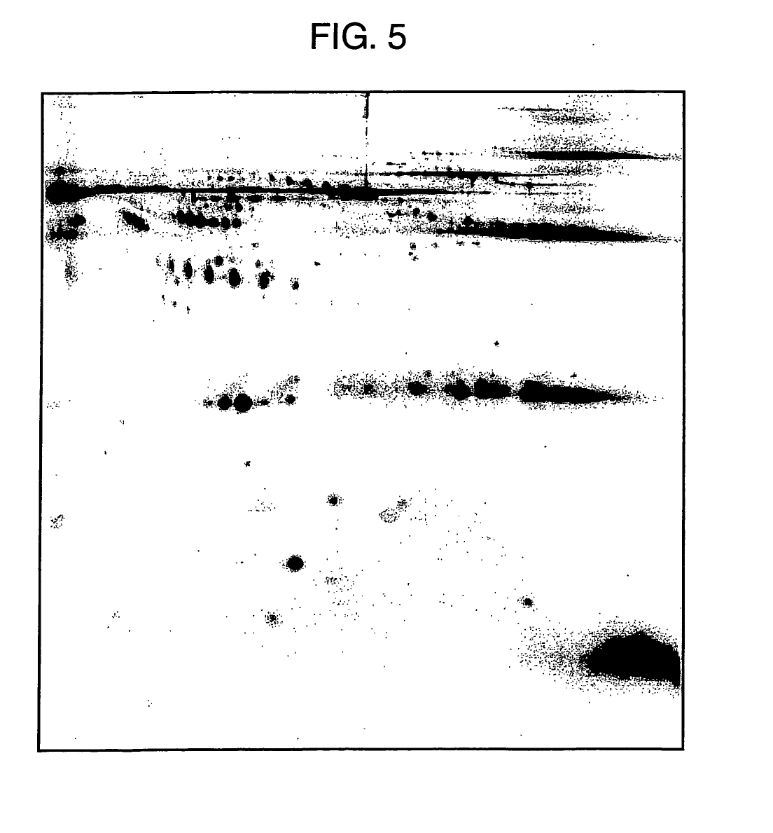

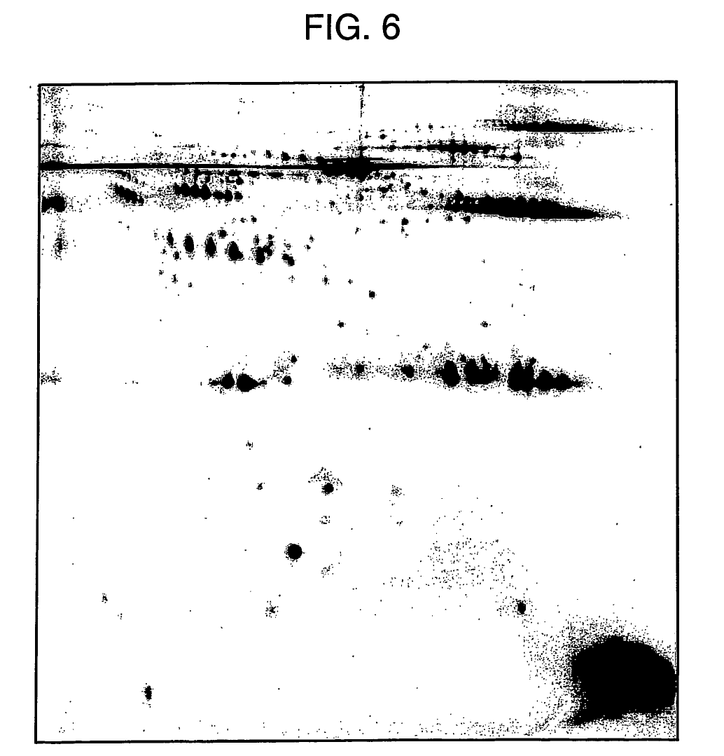

Comparing the plasma proteome from zero (FIG. 5) to 14 days (FIG. 6) at −80° C. indicates that the great majority of proteins are preserved during freezing. A small number of low abundance spots appear to be present only after storage. The total protein on both gels appears very similar.

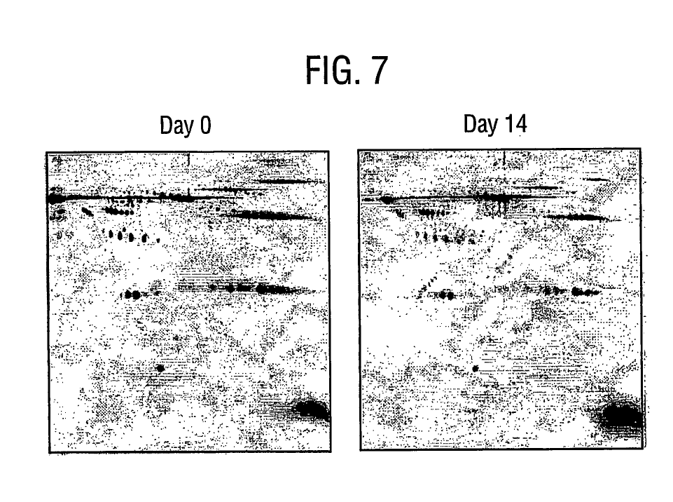

Comparing the plasma proteome from zero to 14 days (FIG. 7) stored on the PVA-treated 31ETF paper indicates that the great majority of proteins are also preserved during storage on this media. Again, a small number of low abundance spots appear to be present only after storage, and these extra spots are identical to the extra spots that appear after storage at −80° C. There appear to be very few differences in the 14 day proteomes of the plasma which was stored at −80° C. and the plasma which was stored on the PVA-treated 31ETF paper, except that the total protein on the gel appears to be generally slightly reduced in the gel corresponding to the plasma stored on the PVA-treated 31ETF paper compared to the zero time gel.

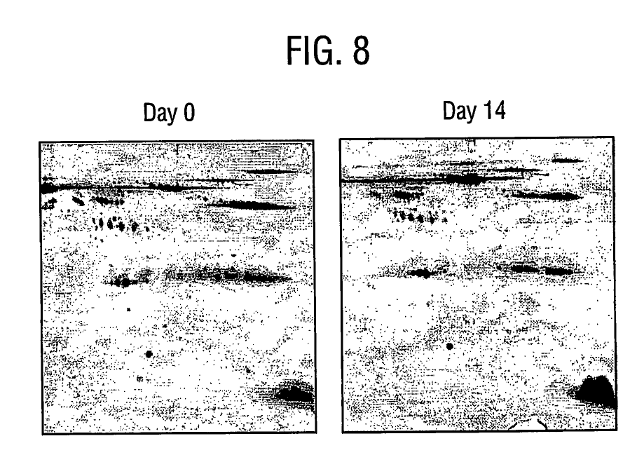

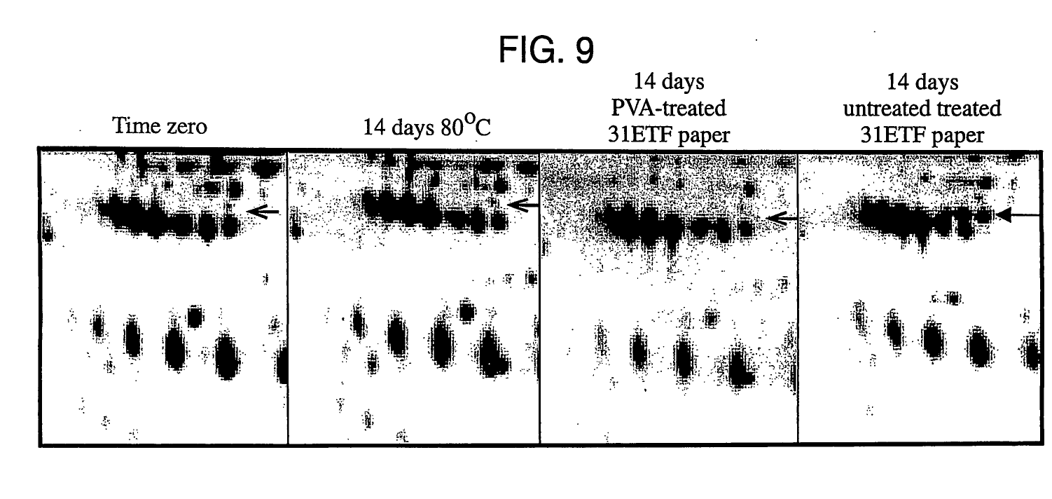

The plasma proteome from 14 day non-PVA treated 31ETF paper (FIG. 8) also appeared very similar to that of the frozen plasma. Again, the total protein on the gel appeared slightly reduced in the gel corresponding to the 31ETF paper compared to the zero time gel. A peculiarity of the gel corresponding to the non-PVA treated 31ETF paper is shown in FIG. 9, with the appearance of a number of abundant protein spots running just above the α-1 antitrypsin charge-train.

### SUMMARY AND CONCLUSIONS

We have demonstrated that 31lETF paper is capable of preserving the proteome of human plasma over a period of 14 days at room temperature. The gels produced from proteins extracted from the PVA-treated 31ETF paper are effectively identical to those from serum that had been stored at −80° C. for 14 days.

A small number of differences were noted after 14 days between the gels corresponding to the non-PVA treated 31ETF paper and those that had been stored −80° C. for 14 days, although the great majority of spots were unchanged.

A general reduction in the amount of protein on the gel was observed from both types of 31ETF paper but as this appeared to be a general reduction in total proteins -this could be counteracted by increasing the amount of protein initially spotted onto the 31ETF discs.

For the application of preserving the human plasma proteome we would recommend that PVA-treated 31ETF paper be used in preference to non-PVA-treated 31ETF paper.

Over 500 distinct protein spots were detected in the plasma protein. The vast majority of these proteins migrated to the same position of isoelectric point and molecular weight after 14 days storage on PVA-treated 31ETF paper indicating that they had been preserved in their in vivo state during storage.

### Example 4

We have recently commenced a long-term stability study under controlled conditions of temperature and humidity (4° C., ambient humidity; 20° C., 55% RH, 86% RH; 40° C., 53% RH, 88% RH). In each case we have stored a solution containing 400 U/ml alkaline phosphatase (10 μl) in PBS and porcine pancreatic trypsin (Sigma Proteomics Grade), 5 μg/ml in 1 mM HCl (10 μl) on PVA-treated 31ETF. We consider that no significant loss (<25%) of enzymatic activity have been observed after 6 months at 4° C. and 20° C. but at 40° C. losses would appear higher and are considered significant (>25%). In a parallel study we artificially aged PVA treated Whatman 31ETF paper (smooth cellulose) by 10 years (3 days at 82° C. and 65% RH) before applying and storing proteins as above. Results are similar for these aged samples as for new. It is not feasible to artificially age protein-loaded material, as the extreme conditions would likely denature proteins.

### Example 5

Since trypsin is used routinely in the digestion of protein spots for example after 2D-GE the utility of 250 ng stably stored trypsin on 3% (w/v) polyvinylalcohol (PVA;GLO3) treated Whatman 31ET paper (smooth cellulose) for tryptic digestion of protein spots obtained by 2D-GE of human heart left ventricle was assessed.

Briefly the protein extract was isoelectric focused using IPG strips (AmershamBioscience), with pH range 3-10 (non-linear), using an in-gel rehydration method. The samples were diluted with rehydration solution prior to rehydration overnight in a reswelling tray. Total protein loads were 400 μg. After IEF the strips were equilibrated in equilibration buffer with 1% DTT for 15 minutes, followed by the same buffer with 4.8 % iodoacetamide for 15 minutes. SDS-PAGE was performed using 12% T, 2.6% C separating gels without a stacking gel using a Hoefer DALT system. The second-dimension separation was carried out overnight and was stopped as the dyefront just left the bottom of the gels. All gels were fixed and stained using the PlusOne Silver Staining Kit (Amersham Bioscience, UK).

Two sets of four spots were cut from duplicate preparative-scale 2D-PAGE gels. The spots chosen were of medium-high abundance on the gels. Each spot was de-stained to remove silver ions, washed extensively and dried down in a centrifugal evaporator. The two sets of spots were then treated as follows: Set 1 had 250 ng of trypsin added that was freshly made up in ammonium bicarbonate buffer (total volume, 10 μl ). Set 2 had one disc of trypsin (250 ng) on the PVA-treated paper added per spot and 10 μl of ammonium bicarbonate buffer added. All spots were allowed to re-hydrate and excess trypsin was not removed from the spots. Trypsinolysis occurred at 37° C. for 6 hours.

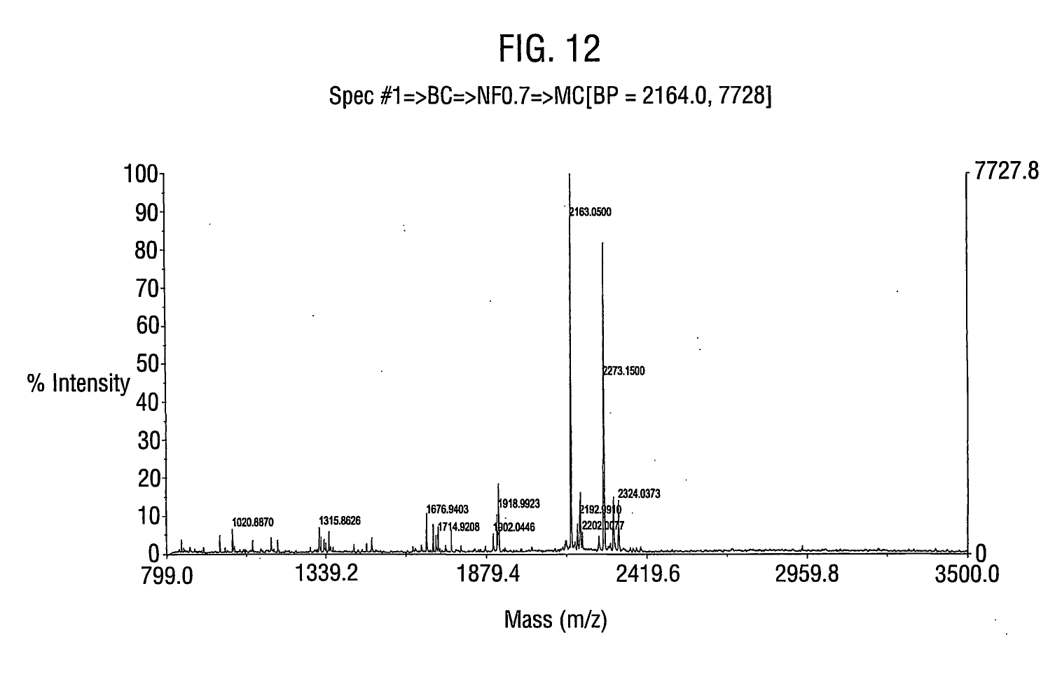

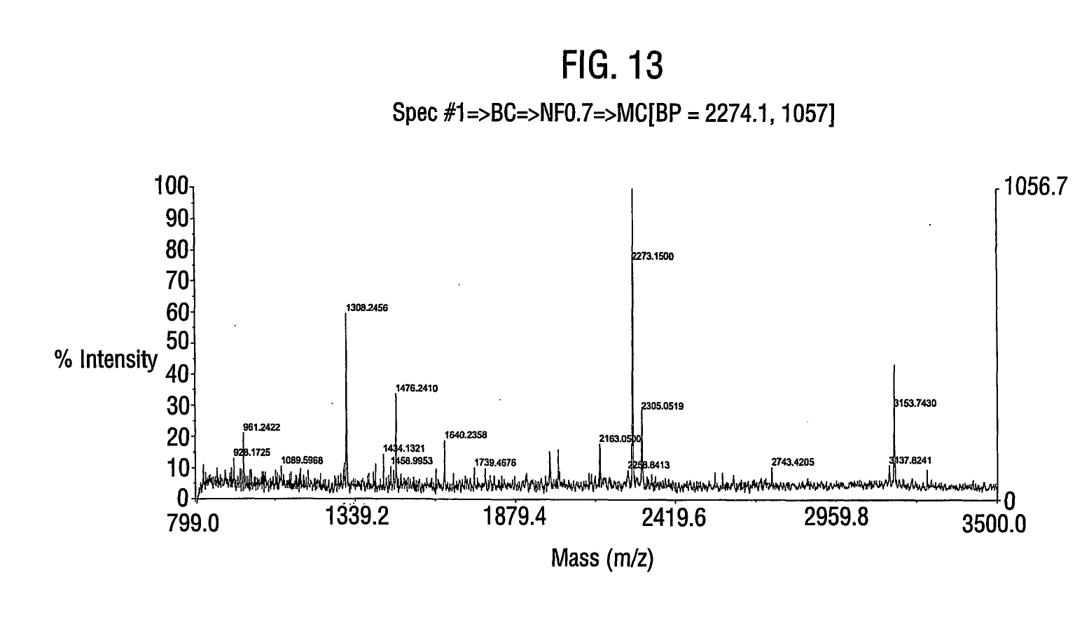

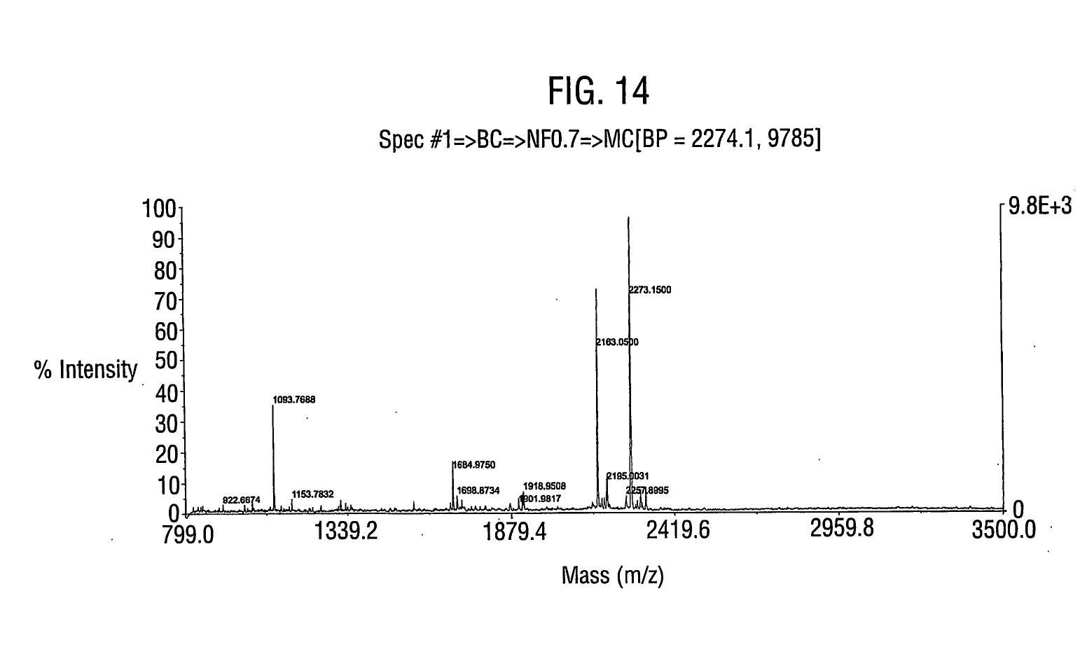

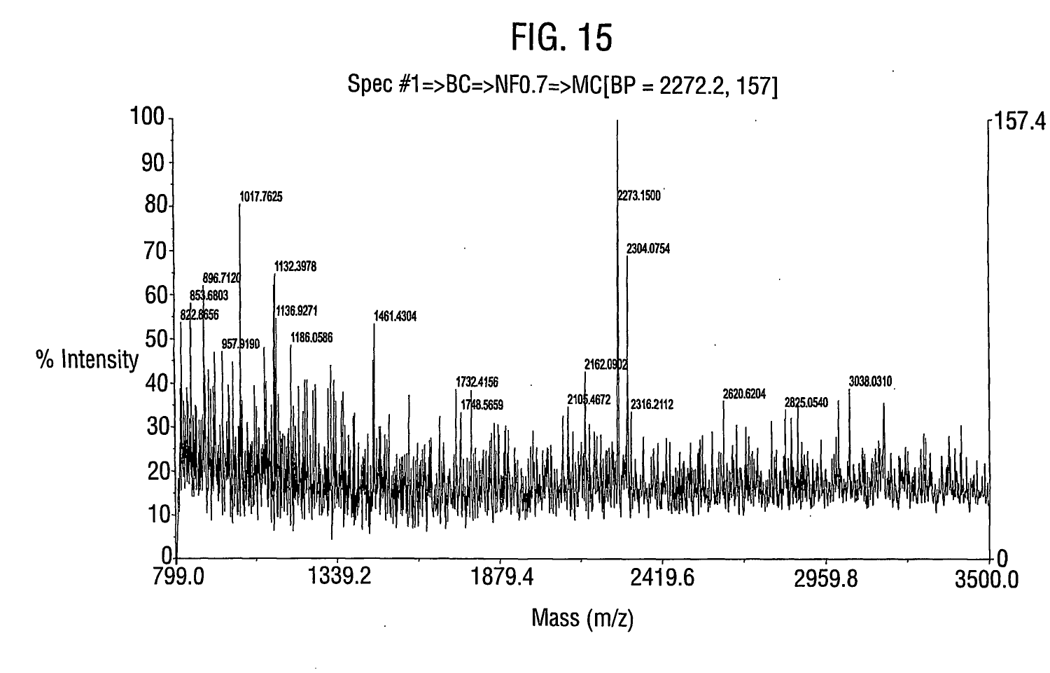

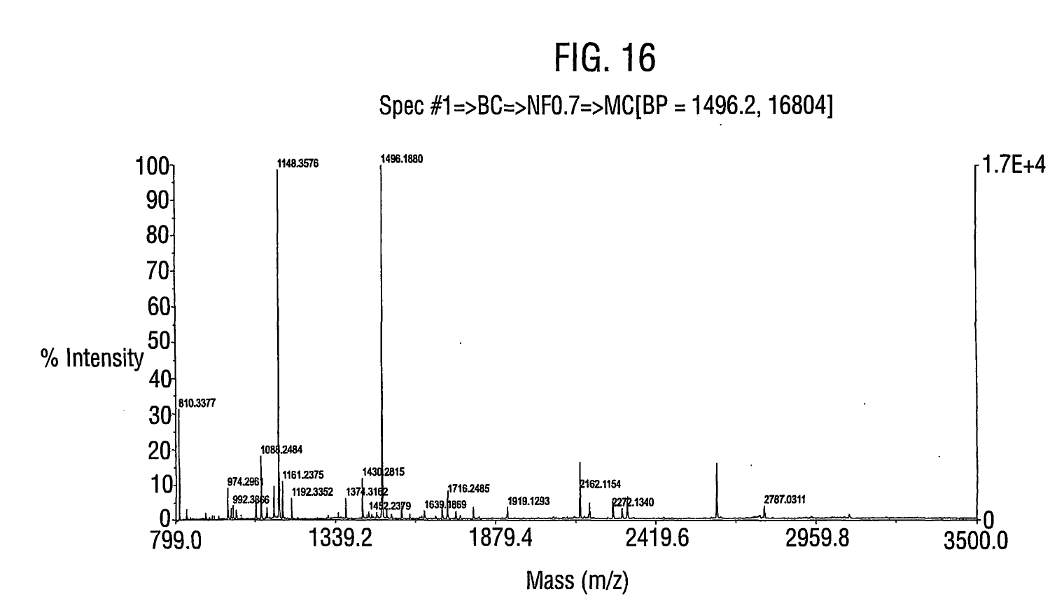

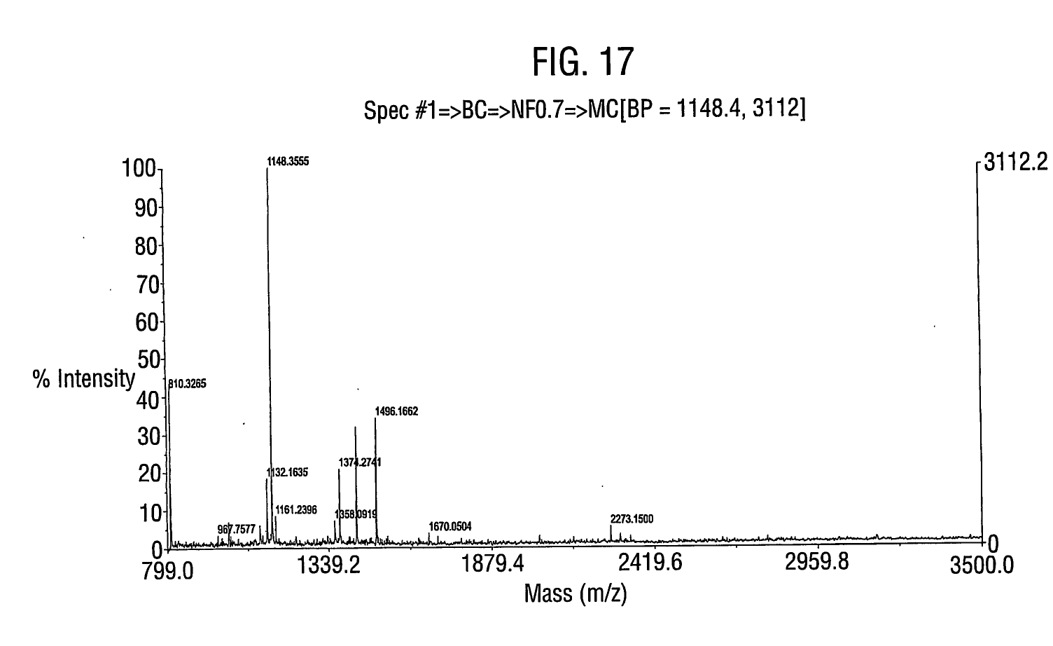

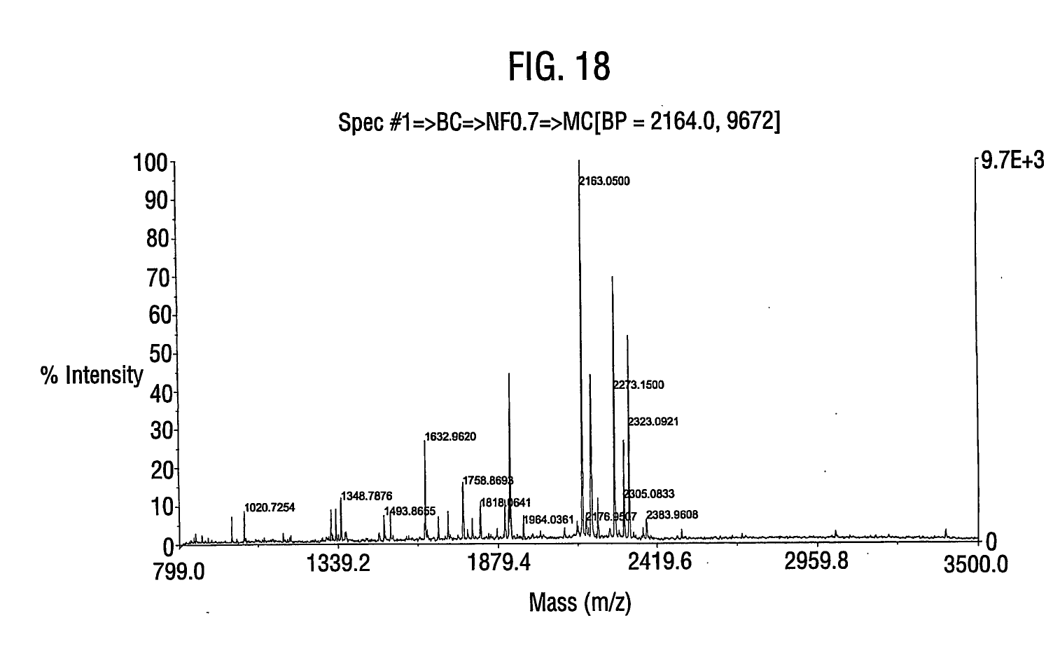

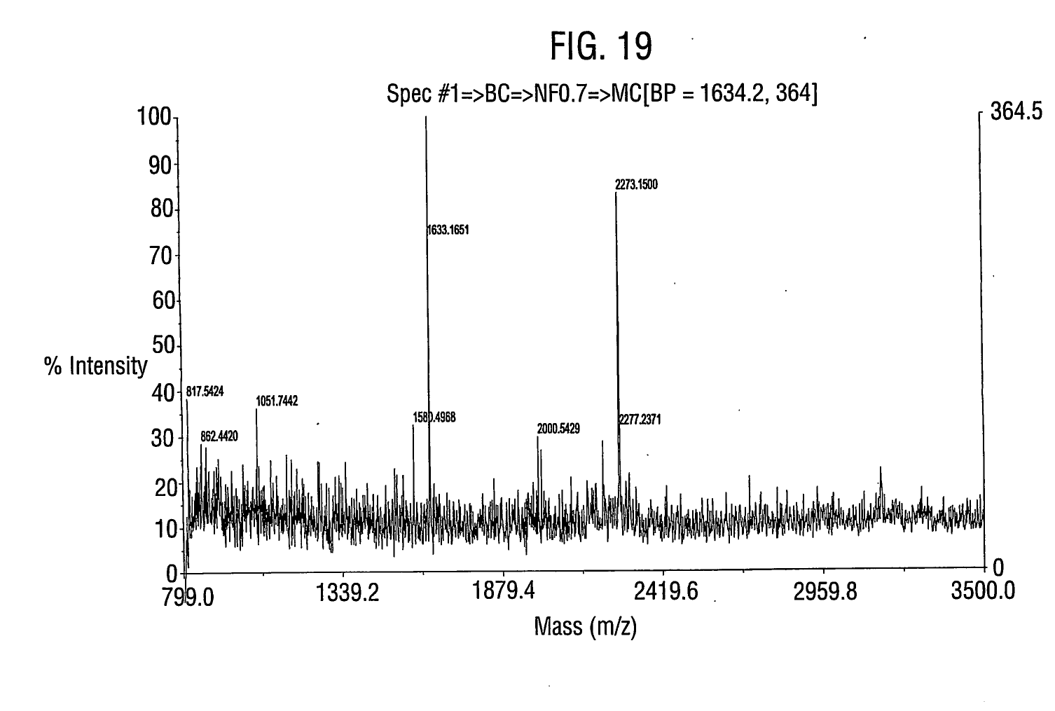

Liberated peptides were cleaned, de-salted and concentrated using ZipTips (Millipore). The purified peptides were lyophilized in micro-Eppendorf tubes using a centrifugal evaporator. All peptide mixtures were mixed with matrix (αacyano-4-hydroxycinnamic acid) and spotted onto the Voyager DE Pro target plate. After drying the plate was inserted into the MS and MALDI-TOF spectra obtained for each peptide mixture. The spectra for each spot digest are presented in FIGS. 12 to 19.

The presence of trypsin autolytic fragments, together with peptides originating from the digested proteins in every sample indicated that trypsin activity was present on and elutable from the PVA-treated paper discs. These peaks were in general significantly reduced in the samples digested using trypsin stored on the PVA-treated paper. Trypsin autolytic peptides are produced during trypsinolysis and originate from the intermolecular proteolysis of trypsin molecules. Sigma's bovine trypsin typically produces autolytic peptides with molecular masses of 2163.05 Daltons (residues 50-69) and 2273.15 Daltons (residues 70-89), which were present in every sample. In Spots 2 and 3 for example these peaks are orders of magnitude lower in the PVA-treated paper samples relative to the freshly made up trypsin samples. In general the higher the concentration of trypsin used for the digestion of a spot, the more prominent these autolytic peptides become. In the digestions described here we freshly made up trypsin was added at the same concentration to that expected to have eluted from the PVA-treated paper disc (250 ng in 10 μl ). It would appear therefore that the amount of trypsin obtained from the PVA-treated paper discs was substantially lower than 250 ng. This is perhaps anticipated since there will be a proportion of the trypsin solution still retained in the body of the PVA-treated paper disc and this would not necessarily be able to hydrolyse proteins as efficiently as totally liberated trypsin molecules.

### Example 6

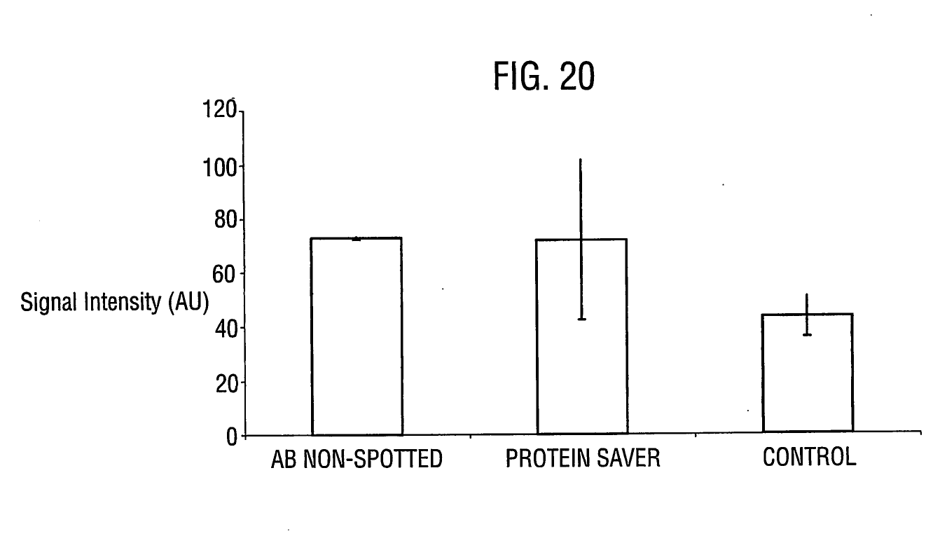

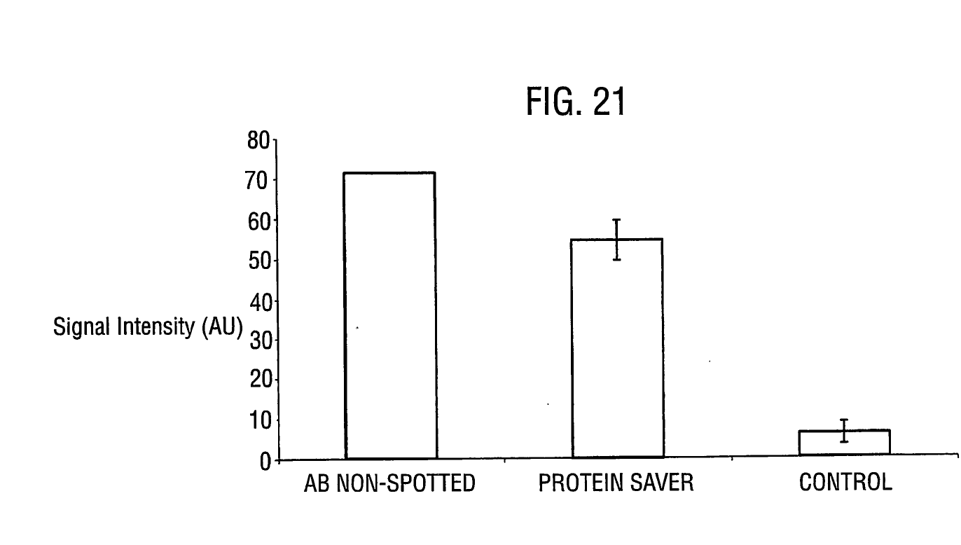

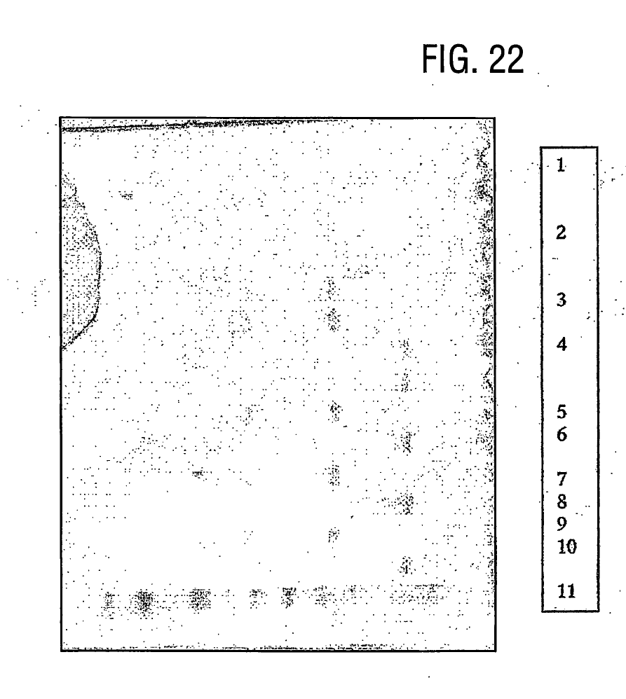

Purified C595 anti-MUC1 murine IgG 3 monoclonal antibody (50 μ; 1 mg/ml in PBS) was applied to 3% (w/v) polyvinylalcohol (PVA;GLO3) treated Whatman 31ET paper (smooth cellulose) and a control substrate and allowed to air-dry for 1 hour. Samples were stored at room temperature for 28 days. Antibody was eluted by incubation with PBS (1 ml) for 30-45 mins. Samples of eluate were assayed by ELISA (FIGS. 20 and 21) and SDS-PAGE (FIG. 22).

Data obtained from this study indicate stable storage and recovery of monoclonal antibody following storage at room temperature for 28 days. The antibody retained immunoreactivity at both the antigen binding site as well as the binding site for the secondary anti-murine antibody.

### Example 7

Protein Mixtures

Tissue Culture Supernatant

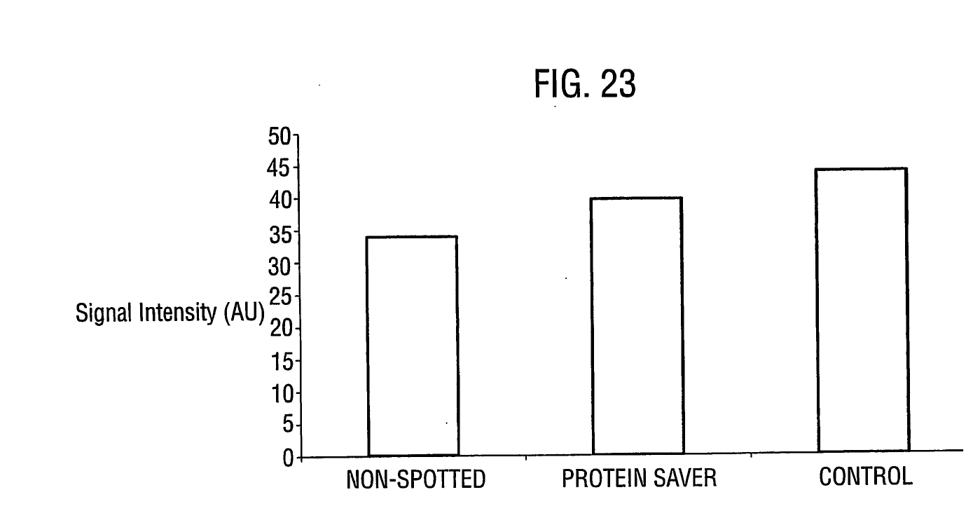

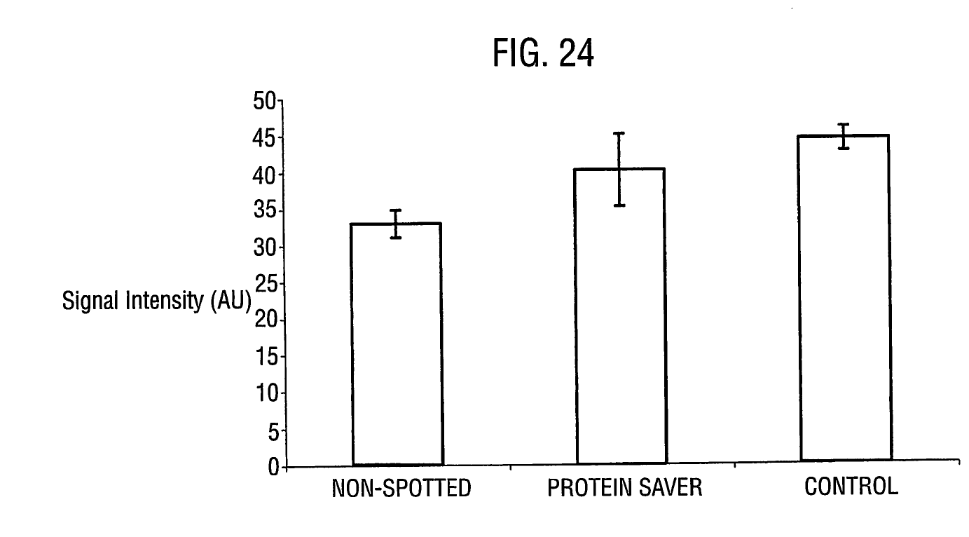

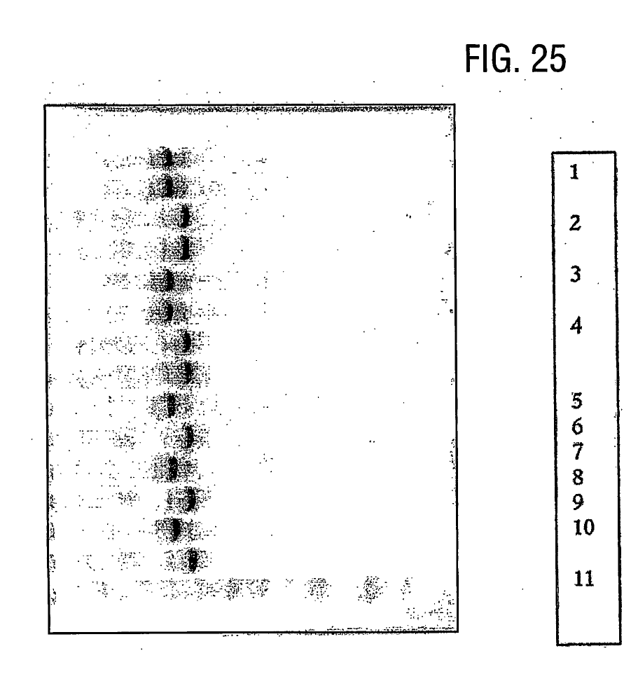

Tissue culture supernatant (50 μl) in which murine hybridoma cells (C595/102) expressing C595 anti-MUC1 murine monoclonal antibody had previously been grown was applied to 3% (w/v) polyvinylalcohol (PVA;GLO3) treated Whatman 31ET paper (smooth cellulose) and a control substrate and allowed to air-dry for 1 hour. Samples were stored at room temperature for 28 days. Proteins were eluted by incubation with PBS (1 ml) for 30-45 mins. Samples of eluate were assayed by ELISA (FIGS. 23 and 24) and SDS-PAGE (FIG. 25).

The data indicate that tissue culture supernatant based on RPMI 1640 medium supplemented with 10% (v/v) heat inactivated foetal calf serum and 2 mM glutamine could be stored on the PVA-treated paper for at least 28 days under ambient conditions. Proteins were detected by 1D-GE and also the presence of the monoclonal antibody by ELISA. It was evident from the data that the control substrate could also store and elute proteins but these studies and their assays do not provide sufficient information to discriminate relative protein profiles, concentrations or biological activities between each substrate.

Human Plasma

See Example 3.

Peptide Mixture Following Tryptic Digestion of a Protein

Bovine serum albumin (BSA) was diluted in 25 mM ammonium bicarbonate solution to a final concentration of 8 mg/ml. Bovine trypsin was then added to a final ratio of 1:50 enzyme:substrate. Digestion was carried out overnight at 37° C. The initial digestion mixture was stored as follows:
6× 10 ul applied to 3% (w/v) polyvinylalcohol (PVA;GLO3) treated Whatman 31ET paper (smooth cellulose), (stored at room temperature, 20° C.)
6× 10 ul applied to Control substrate (stored at room temperature, 20° C.)
4× 20 ul aliquots into Eppendorf tubes (2 stored at 20° C., 2 stored at 4° C.).

After storage for 7days the samples were taken out of storage and, with a fresh digest, prepared for MALDI analysis. The digestion mixture was eluted from the PVA-treated. paper and the Control substrate by the addition of 1 ml of water (due to the high concentration of the digest) and incubated at room temperature for 15 minutes with occasional agitation. The eluted peptides were assumed to be at a concentration of 80 ng/ul, and all other digestion solutions were diluted accordingly.

Samples were applied to the MALDI target by the “dried-drop” method. 1 μl of sample was mixed with 1 μl of 4 mg/ml α-cyano-4-hydroxycinnamic acid in 60:40 ACN:H₂O+0.1% TFA and spotted directly onto the target. Spectra were captured using a Micromass reflectron bench-top MALDI.

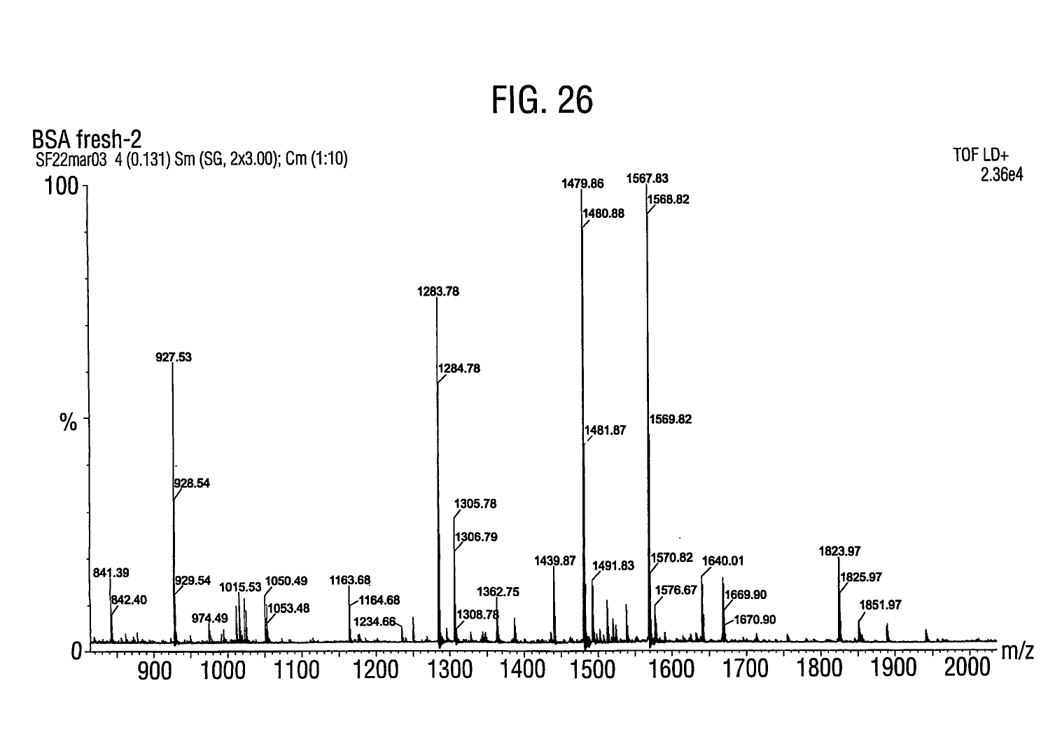

A spectrum obtained from the fresh digest of BSA is shown in FIG. 26.

In the fresh digest 22 peptides were identified which covered 40% of the BSA sequence. There was no evidence of trypsin or keratin contamination. Unidentifiable peaks are assumed to be contaminants and, with exception of m/z 1234.66, 1491.83 and 1576.67, these peaks were close to background noise and were therefore considered insignificant

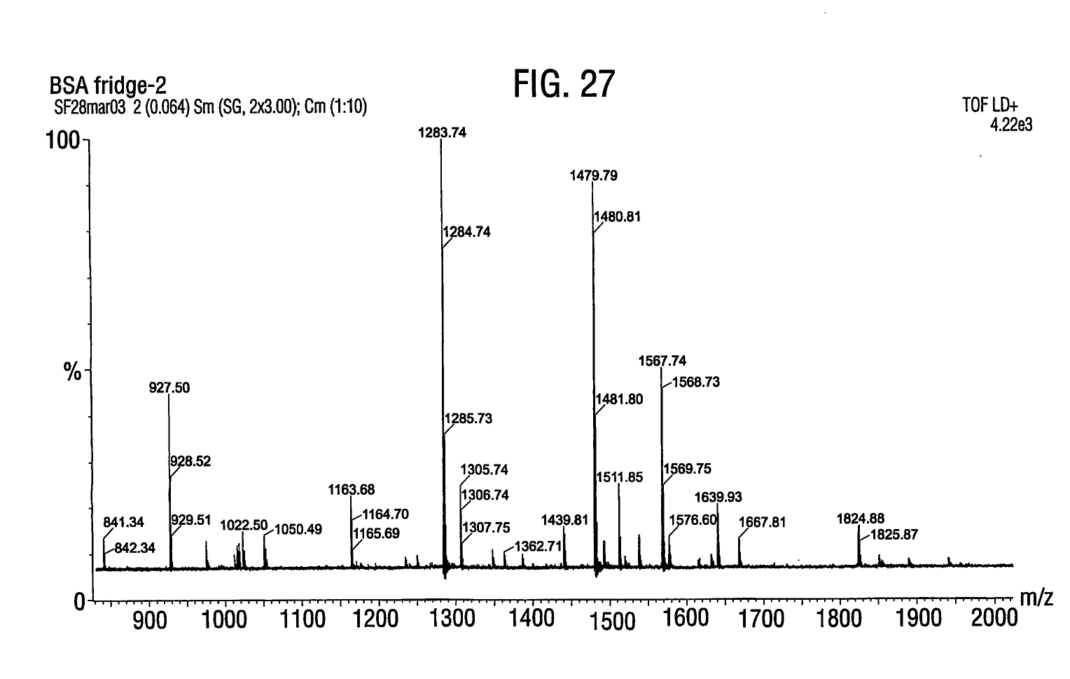

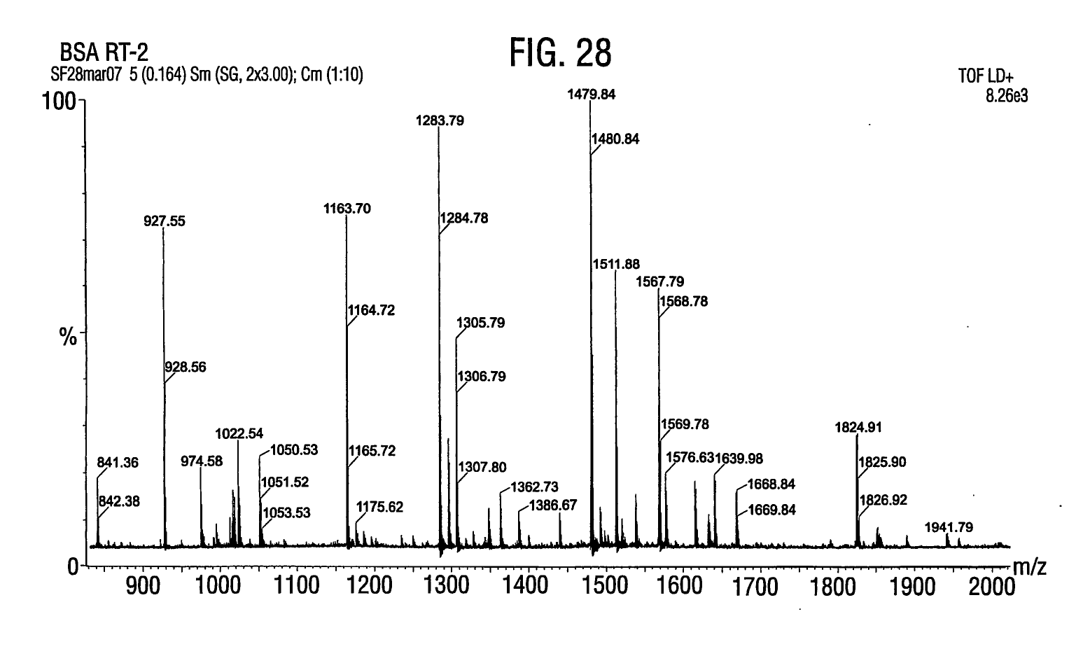

Spectra obtained from the aliquots of digested BSA following storage for 7 days at either 4° C. or 20° C. are shown in FIGS. 27 and 28 The digest after storage at 4° C. identified 19 peaks, although 2 (m/z 1249.62 and 1850.90) are very weak. Peaks m/z 1011.42, 1519.75 and 1888.93 are missing. The digestion profile is similar to that observed for the fresh digest, although the overall signal intensity has diminished (4e³). The digest after storage at 4° C. identified 18 peaks, with 1011.42, 1249.62, 1519.75 and 1888.93 missing. There is notable increase in the level of background noise in the spectrum, with respect to a fresh digest although the majority of these peaks have a relative intensity of less than 5%. In addition to the previously identified contaminants in the fresh digest, peaks of m/z 1022.54, 1175.62 1295.76, 1347.60, 1624.65, 1631.71 have been identified. None of these peaks correspond to trypsin, and they are assumed to be the result of sample degradation. Again, the intensity of some peaks increases (e.g. n/z 1163.70) and decreases (e.g. m/z 1567.79) with respect to the fresh digest.

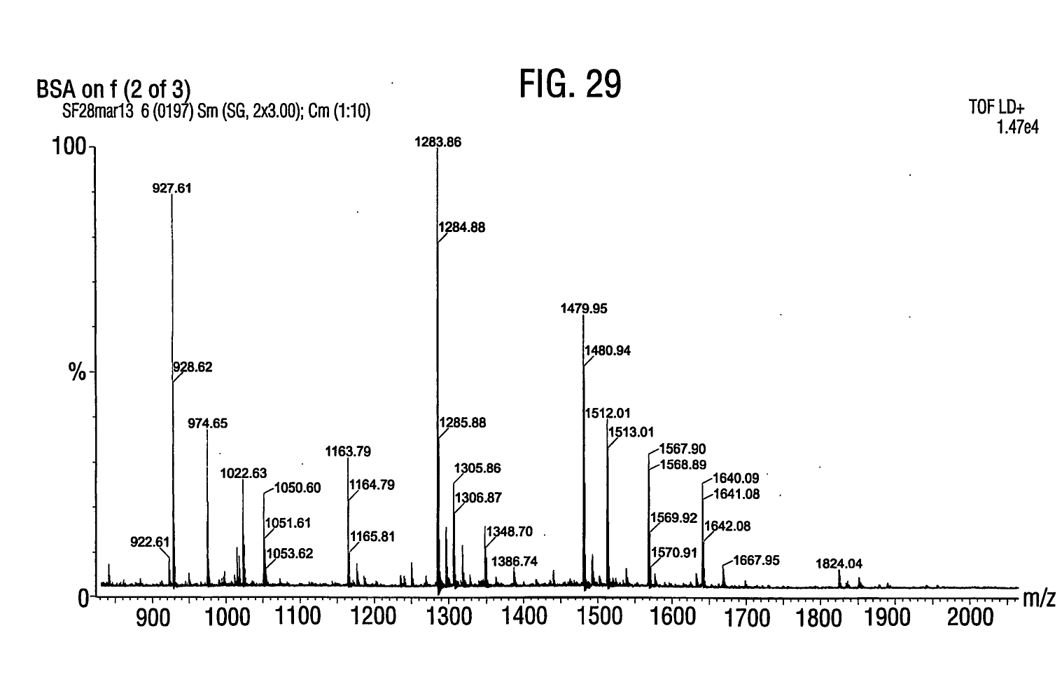

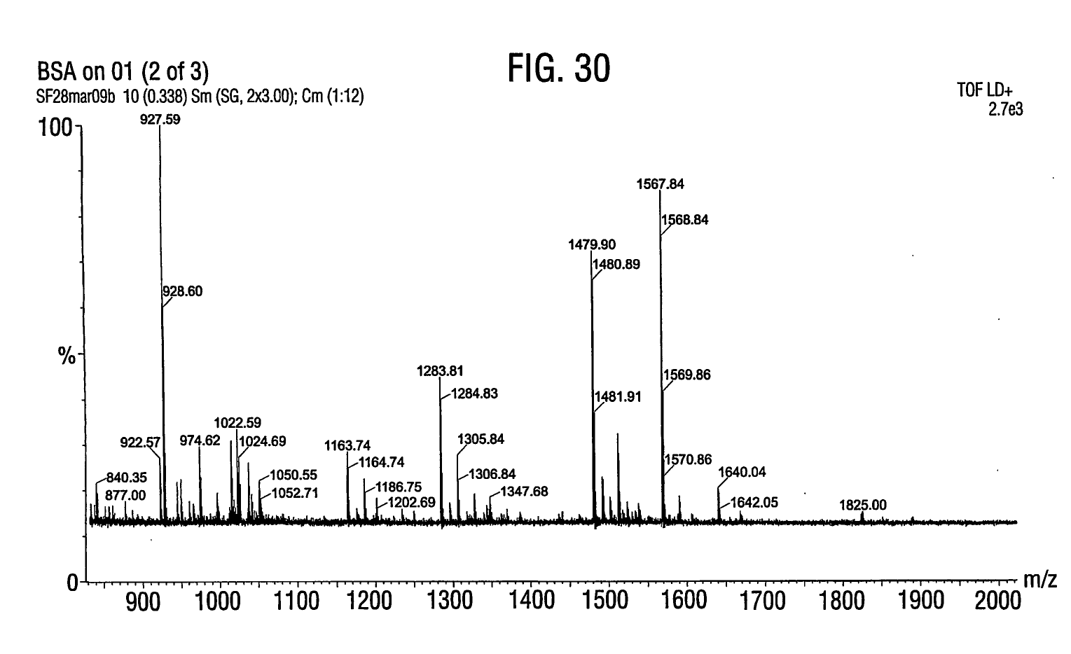

The spectra obtained from digested BSA following storage for 7 days on PVA-treated paper or the Control substrate at 20° C. are shown in FIGS. 29 and 30. The sample stored on PVA-treated paper identified 18 peaks with m/z 1011.42, 1362.67, 1519.75 and 1888.93 missing. The overall digestion profile is very similar to that of the 7 days 20° C. digest although there is less contamination evident. The Control substrate identified 14 peaks with m/z 1011.42, 1249.62, 1362.67, 1386.62, 1439.81 1519.75, 1850.90 and 1888.93 missing.

### CONCLUSION

In this report we have summarised the data obtained on protein storage using polyhydric-treated substrates. Several key conclusions may be drawn:
1) The list of proteins or mixtures tested is not exhaustive and these data merely illustrate the possibilities of this material.
2) Aside from pre-purified proteins it has been impractical to determine the biological activity of components of protein mixtures following storage on the polyhydric-treated substrates of the invention.
3) No specialised buffer systems were required for protein loading.
4) The physical event occurring with the polyhydric-treated substrates of the invention is evaporation
5) No specialised buffer systems were required for protein elution. It should be noted that non-volatile buffer salts will be retained on the substrate during evaporation and these may redissolve during elution. Consequently water or dilute buffer may be an appropriate eluent.
6) The volume of eluent typically exceeds the sample volume hence the eluted material may be more dilute than the original sample.
7) Recovery of eluted protein is considered to be >75% (w/w). A proportion of the eluted protein remains within the mobile phase associated with the rehydrated polyhydric-treated matrix and is effectively unrecoverable. Presumably this proportion reduces as eluent volume increases.

1. A method of stably storing a protein, the method comprising applying a protein to be stored to a cellulose substrate which has been treated with a polyhydric compound and dried, wherein the amount of the polyhydric compound present in the substrate is sufficient to stabilize the activity of the protein or to chemically stabilize the protein, and wherein the substrate does not consist of glass.

2. (canceled)

3. (canceled)

4. A method of stably storing a protein, the method comprising applying a protein to be stored to a substrate which has been treated with a polyhydric compound and dried, wherein the amount of the polyhydric compound present in the substrate is sufficient to stabilize the activity of the protein or to chemically stabilize the protein, and wherein the substrate does not consist of glass and wherein the polyhydric conpound is polvinylalcohol (PVA) having a degree of hydrolysis of less than about 97.5%, a molecular weight of 9000-186000 or both.

5. The method of claim 1, wherein a blood protein is stored.

6. (canceled)

7. The method of claim 1, wherein the proteome of whole blood or of a blood fraction is stored.

8-10. (canceled)

11. The method of claim 4, wherein the substrate comprises cellulose fibers.

12. The method of claim 4, wherein the substrate consists of cellulose fibers.

13. (canceled)

14. The method of claim 1, wherein the polyhydric compound is selected from the group consisting of: a PVA having a molecular weight of 9000-186000 and a degree of hydrolysis of at least 70%; glycerol; sucrose; carrageenan; xanthan gums; and pectin.

15. The method of claim 1, wherein the polyhydric compound is a PVA having a degree of hydrolysis less than about 97.0%.

16. The method of claim 1, wherein the polyhydric compound is a PVA having a degree of hydrolysis of between 84% and 92%.

17. The method of claim 1, wherein, following storage of the protein on the substrate, the protein is eluted from the substrate for subsequent use.

18-20. (canceled)

21. The method of claim 1, wherein the protein to be stored has been subjected to chromatographic purification.

22. (canceled)

23. The method of claim 1, wherein the protein to be stored is a proteinase.

24. (canceled)

25. A method of digesting a protein, wherein the method comprising:
(a) stably storing a proteinase on a substrate to thereby obtain a proteinase-loaded substrate, the proteinase being stored by a method comprising applying the proteinase to a cellulose substrate which has been treated with a polyhydric compound and dried, wherein the amount of the polydydric compound present in the substrate is sufficient to stabilize the activity of the proteinase, and wherein the substrate does not consist of glass; and
(b) applying the protein to be digested to the proteinase-loaded substrate under suitable conditions for the digestion of said protein to thereby effect digestion of the protein; or
eluting the proteinase from the proteinase-loaded substrate to obtain a proteinase solution which is then used to effect digestion of the protein.

26-32. (canceled)

33. A proteome stored on a cellulose substrate which has been treated with a polyhydric compound and dried, wherein the amount of the polyhydric compound present in the substrate is sufficient to stabilize the activity of the proteins of the proteome or to chemically stabilize the proteins of the proteome, and wherein the substrate does not consist of glass.

34. A proteome stored on a substrate which has been treated with a polyhydric compound and dried, wherein the amount of the polyhydric compound present in the substrate is sufficient to stabilize the activity of the proteins of the proteome or to chemically stabilize the proteins of the proteome, and wherein the substrate does not consist of glass and wherein the polvdric compound is polyvinylalcohol having a degree of hydroloysis of less than about 97.5%, a molecular weight of 9000-186000, or both.

35-41. (canceled)

42. The method of claim 4, wherein a blood protein is stored.

43. The method of claim 4, wherein the proteome of whole blood or of a blood fraction is stored.

44. The method of claim 4, wherein the polyhydric compound is a PVA having a degree of hydrolysis of between 84% and 92%.

45. The method of claim 4, wherein, following storage of the protein on the substrate, the protein is eluted from the substrate for subsequent use.

46. The method of claim 4, wherein the protein to be stored has been subjected to chromatographic purification.

47. The method of claim 4, wherein the protein to be stored is a proteinase.

48. A method of digesting a protein, wherein the method comprises:
(a) stably storing a proteinase to thereby obtain a proteinase-loaded substrate, the proteinase being stored by a method comprising applying the proteinase to a substrate which has been treated with a polyhydric compound and dried, wherein the amount of the polyhydric compound present in the substrate is sufficient to stabilize the activity of the proteinase, and wherein the substrate does not consist of glass and wherein the polyhydric compound is polyvinylalcohol having a degree of hydrolysis of less than about 97.5%, a molecular weight of 9000-186000, or both; and
(b) applying the protein to be digested to the proteinase-loaded substrate under suitable conditions for the digestion of the protein to thereby effect digestion of the protein; or
eluting the proteinase from the proteinase-loaded substrate to obtain a proteinase solution which is then used to effect digestion of the protein.
